SEQUENCE LISTING (25 sequences)

SEQ ID NO: 1 - DNA, 22 bp, Artificial
  Synthetic oligonucleotides
aagcagcaagtatgatgagcaa

SEQ ID NO: 2 - DNA, 19 bp, Artificial
  Synthetic oligonucleotides
aagcagcaagtatgatgag

SEQ ID NO: 3 - DNA, 18 bp, Artificial
  Synthetic oligonucleotides
agcagcaagtatgatgag

SEQ ID NO: 4 - DNA, 20 bp, Artificial
  Synthetic oligonucleotides
gaagcagcaagtatgatgag

SEQ ID NO: 5 - DNA, 21 bp, Artificial
  Synthetic oligonucleotides
agctgtgatcctgaaactgaa

SEQ ID NO: 6 - DNA, 23 bp, Artificial
  Synthetic oligonucleotides
gtgatcctgaaactgaattttct

SEQ ID NO: 7 - DNA, 24 bp, Artificial
  Synthetic oligonucleotides
gtgatcctgaaactgaattttcta

SEQ ID NO: 8 - DNA, 25 bp, Artificial
  Synthetic oligonucleotides
gtgatcctgaaactgaattttctat

SEQ ID NO: 9 - DNA, 21 bp, Artificial
  Synthetic oligonucleotides
gtgatcctgaactgaattttc

SEQ ID NO: 10 - DNA, 21 bp, Artificial
  Synthetic oligonucleotides
cagacacatactccataattt

SEQ ID NO: 11 - DNA, 34 bp, Artificial
  Synthetic oligonucleotides
aaaggcattagaaagcctgtagttttacttactc

SEQ ID NO: 12 - DNA, 31 bp, Artificial
  Synthetic oligonucleotides
agaaaggcattagaaagcctgtagttttact

SEQ ID NO: 13 - DNA, 31 bp, Artificial
  Synthetic oligonucleotides
aggcattagaaagcctgtagttttacttact

SEQ ID NO: 14 - DNA, 36 bp, Artificial
  Synthetic oligonucleotides
ctgagaaaggcattagaaagcctgtagttttactta

SEQ ID NO: 15 - DNA, 23 bp, Artificial
  Synthetic oligonucleotides
gtagttttacttactctcgtctc

SEQ ID NO: 16 - DNA, 22 bp, Artificial
  Synthetic oligonucleotides
cgtctccacagacacatactcc

SEQ ID NO: 17 - DNA, 23 bp, Artificial
  Synthetic oligonucleotides
cgtctccacagacacatactcca

SEQ ID NO: 18 - DNA, 25 bp, Artificial
  Synthetic oligonucleotides
cgtctccacagacacatactccata

SEQ ID NO: 19 - DNA, 26 bp, Artificial
  Synthetic oligonucleotides
cgtctccacagacacatactccataa

SEQ ID NO: 20 - DNA, 22 bp, Artificial
  Synthetic oligonucleotides
ctcgtctccacagacacatact

SEQ ID NO: 21 - DNA, 23 bp, Artificial
  Synthetic oligonucleotides
ctctcgtctccacagacacatac

SEQ ID NO: 22 - DNA, 26 bp, Artificial
  Synthetic oligonucleotides
gtctccacagacacatactccataat

SEQ ID NO: 23 - DNA, 27 bp, Artificial
  Synthetic oligonucleotides
gtctccacagacacatactccataatt

SEQ ID NO: 24 - DNA, 195 bp, Homo sapiens
tttctttgaagcagcaagtatgatgagcaagctttctcacaagcatttggttttaaattatggagtatgtgtctgtggagacgagagtaagtaaaactacaggctttctaatgcctttctcagagcatctgtttttgtttatatagaaaattcagtttcaggatcacagctaggtgtcagtgtaaactataattt

SEQ ID NO: 25 - DNA, 195 bp, Homo sapiens
aaagaaacttcgtcgttcatactactcgttcgaaagagtgttcgtaaaccaaaatttaatacctcatacacagacacctctgctctcattcattttgatgtccgaaagattacggaaagagtctcgtagacaaaaacaaatatatcttttaagtcaaagtcctagtgtcgatccacagtcacatttgatattaaa

(19) United States
(12) Patent Application Publication
(10) Pub. No.: US 2007/0224598 A1
(43) Pub. Date: Sep. 27, 2007

(54) System and method for detection of mutations in JAK2 polynucleotides

(75) Inventor: Chung-Che Chang, Bellaire, TX (US)

Correspondence Address: HAYNES AND BOONE, LLP, 901 MAIN STREET, SUITE 3100, DALLAS, TX, 75202, US

(73) Assignee: THE METHODIST HOSPITAL RESEARCH INSTITUTE, Houston, TX (US)

(21) Appl. No.: 11/375,944

(22) Filed: Mar. 15, 2006

(51) Int. Cl. C12Q 1/68; C07H 21/04; C12P 19/34

(52) U.S. Cl. 435/6; 435/91.2; 536/24.3

(57) ABSTRACT

Disclosed are a system and methods for identifying JAK2-specific polynucleotide sequences in a biological sample. Also disclosed are oligonucleotide primer and probe comopositions for detecting mutations in a JAK2 polynucleotides, and the JAK2^{V617F} mutation in specific, as well as systems, diagnostic kits and articles of manufacture comprising JAK2-specific primer and probe compositions.

### BACKGROUND OF THE INVENTION

1.1 Field of the Invention

The present invention relates generally to the fields of molecular biology, and more specifically to clinical laboratory diagnostic methods. In particular, a system, method, compositions, and diagnostic kits are provided for identification, amplification, quantitation and detection of specific mutations in nucleic acid segments that encode mammalian, and in particular, human Janus Nonreceptor Tyrosine Kinase 2 (JAK2) peptides or polypeptides.

1.2 Limitations in the Prior Art

Complexity, turnaround time, instrumentation expense, and a lack of specific amplification and detection primers have all thwarted the use of traditional molecular biological methods such as gene-level analyses and DNA sequencing in clinical and diagnostic screening laboratories for many mutations of clinical significance, including mutations in JAK2-encoding polynucleotides.

At present, none of the traditional methods are practical for cost-effective detection of particular genetic anomalies and mutations in JAK2 DNA segments. At present, no genetic analyses have been commercialized for use in a clinical diagnostic laboratory setting for the detection of specific mutations in polynucleotides that encode mammalian JAK2 polypeptides, and particularly those that encode the JAK2^{V617F} mutation.

### 2. DESCRIPTION OF ILLUSTRATIVE EMBODIMENTS

The present invention overcomes these and other limitations inherent in the prior art by providing a system and method for detecting specific nucleotide mutations in a population of polynucleotides in, or obtained from, a sample, and particularly samples of biological origin. The present invention also overcomes deficiencies in the art by providing a system and method for specifically detecting mutations in polynucleotide sequences that encode a mutated mammalian JAK2^{V617F} polypeptide. The invention provides for the first time, specific oligonucleotide amplification primers and oligonucleotide detection probes, and diagnostic kits comprising them, for detecting JAK2-encoding polynucleotide sequences in general, and polynucleotides encoding the JAK2^{V617F} mutation in specific. The invention also provides amplification primer pairs and detection probe pairs, as well as methods for using such compositions in the preparation of highly-sensitive nucleic acid assay and detection systems that may be exploited to rapidly detect and quantitate these mutations.

Articles of manufacture, including diagnostic kits, that contain such JAK2- and JAK2^{V617F}-specific primers and probes also are provided by the invention. The rapid analyses and increased sensitivity provided using real-time polymerase chain reaction (PCR) assays that employ the compositions of the present invention, particularly when used in concert with the fluorescence resonance energy transfer (FRET)-based detection systems provided herein, illustrate just two of the many advantages the invention provides for diagnosis of JAK2-related disorders, and specifically those attributable to the JAK2^{V617F} mutation, in the clinical laboratory environment.

### 2.1 Myloproliferative Disorders

Myeloproliferative disorders (MPDs) represent a heterogeneous group of clonal stem cell dyscrasias characterized by abnormal hematopoetic cell proliferation with relatively normal differentiation and maturation. Depending on the specific lineage affected, MPDs may manifest as polycythemia vera (PV), essential thrombocytosis (ET) or idiopathic myelofibrosis (MF). Although precise diagnostic criteria based upon the coexistent presence of multiple nonspecific findings have been defined for each entity, the lack of any one distinct clinical or morphological feature makes them difficult to distinguish from reactive bone marrow conditions.

### 2.2 Janus Nonreceptor Tyrosine Kinase 2 Mutations

Several reports have described a specific mutation in DNA sequences encoding the Janus Nonreceptor Tyrosine Kinase 2 (JAK2) polypeptide that results in a valine to phenylalanine substitution at codon 617 of the JAK2 polypeptide. This mutation, often abbreviated “JAK2^{V617F}”, occurs in a significant proportion of patients who are subsequently diagnosed with one or more myeloproliferative disorders. The JAK2^{V617F} mutation (which occurs exclusively in the dyscratic granulocyte precursor) has been identified in about 65-97% of PV, 23-57% of ET and 35-57% of MF specimens¹⁻⁶. This mutation has also been observed in several related leukemic disorders, including 33% of chronic neutrophilic leukemia, 2% of chronic eosinophic leukemia and 20% of chronic myelomonocytic leukemia⁷. Importantly, acquisition of JAK2^{V617F} has never been identified in healthy controls or in patients known to have unrelated bone marrow disorders⁸⁻¹¹. These findings strongly indicate that the presence of JAK2^{V617F} is of particular clinical significance for diagnosising myeloproliferative disorders, and particularly in situations when the clinical and/or morphological examinations are equivocal. Furthermore, quantitation of the amount of granulocytic cells with JAK2^{V617F} may also serve as an important marker of disease progression and/or treatment response.

Despite their diverse clinical presentation, polycythemia vera, essential thrombocytosis and idiopathic myelofibrosis have been traditionally classified in a common group termed the myeloproliferative disorders (MPD). This system has been the subject of much ongoing debate¹⁴, and although precise diagnostic criteria have been defined for each entity, the lack of characteristic morphological or cytogenetic features makes their distinction from reactive conditions difficult^{15, 16}.

To date, all reported assays to identify the JAK2^{V617F} mutation have utilized various DNA sequencing platforms to identify the presence of the mutated allele in purified granulocyte fractions of peripheral blood or bone marrow aspirate. Although this technique is valid for the detection of such mutated polynucleotides, DNA sequencing is a cumbersome, expensive, and time-consuming technique. As such, while DNA sequencing may be useful in the research laboratory and in academic pursuits of the molecular analyses of JAK2 mutations, it is poorly suited for routine use in clinical diagnostic laboratories.

To overcome limitations in the prior art, the invention provides for a real-time PCR assay that can be readily integrated into a clinical setting. Specific primers and hybridization probes have been developed that distinguish the wildtype and mutant JAK2 alleles by melting curve analysis. This assay has been shown to be a rapid, highly-reliable method for identifying and semi-quantitatively measuring the presence or absence of JAK2^{V617F} from control cell lines, unfractionated peripheral blood, bone marrow specimens, as well as paraffin-embedded formalin-fixed bone marrow clot sections.

Illustrative embodiments of the invention are described below. In the interest of. clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### 2.3 Oligonucleotide Compositions

In one embodiment, the present invention provides oligonucleotide probes and primer sequences specific for mammalian JAK2-encoding polynucleotides that are useful in hybridization to, and amplification of, corresponding homologous JAK2 polynucleotide sequences. In illustrative embodiments, exemplary oligonucleotide primer sequences are disclosed that are useful in the detection and amplification of particular genetic mutations in a JAK2 nucleic acid sequence. In additional embodiments, exemplary oligonucleotide FRET detection probe sequence are disclosed that are particularly useful in the detection and quantitation of amplification products arising from mammalian JAK2^{V617F}-specific polynucleotides. Detection of these products when indicative of the presence of these JAK2^{V617F}-specific polynucleotides in a clinical sample can provide clinical diagnosticians and other medical professionals with a means for predicting and/or confirming the likelihood of particular MPDs, in patients from whom such samples are collected. Such information may also be useful in the management of care for such individuals, and may also serve as molecular markers for determining the extent, significance, and/or rate of disease progression.

The oligonucleotide primers and probes of the present invention are designed for the selective amplification and detection of mammalian JAK2-encoding nucleic acid segments, and JAK2^{V617F}-encoding polynucleotides in particular. The disclosed primer sequences are suitable for use in hybridization methods, and in DNA amplification methods such as PCR-based amplification methods (including, for example, real-time PCR analyses). Likewise, the disclosed oligonucleotide detection probes are suitable for labeling with an appropriate label means for detection and quantitation of the products resulting from the amplification of nucleic acids using one or more pairs of the amplification primers disclosed herein.

When labeled with appropriate markers, the oligonucleotide detection probes are particularly suited for fluorescence-based detection, including, for example, FRET-based analyses. The FRET-labeled detection probe pairs disclosed herein are particularly useful in fluorimetric detection methodologies, including for example, the FRET-based microvolume fluorimetry devices. Use of one or more of the disclosed amplification and detection oligonucleotides pairs is particularly contemplated in the combined real-time PCR/microvolume fluorimetry FRET-based methodologies (real-time PCR-FRET), and particularly in analyses facilitated by the “LightCycler®” instrumentation as development by Idaho Technology, Inc. and now manufactured and marketed by Roche Molecular Systems as described in more detail hereinbelow.

In general, the oligonucleotide probes and primers finding particular utility in the practice of the disclosed methods should be of sufficient length to selectively hybridize to a complementary nucleic acid sequence, such as for example, a region of genomic DNA from a mammalian patient that is suspected of comprising a JAK2 nucleic acid sequence. In particular, oligonucleotide primers and probes are selected such that the selectively hybridize to specific complementary nucleic acid sequences upstream and downstream of a region of DNA that encompasses a sequence that encodes a mutated JAK2^{V617F} polypeptide. The selection of oligonucleotide probe and primer lengths is a process well-known in the molecular biological arts, and depends upon a number of parameters.

For most embodiments, the inventor contemplates that the length of the selected probe and primer compositions of the invention will preferably be less than about 50 to 60 or so nucleotides in length, and more preferably, will be less than about 40 to 45 or so nucleotides in length, while other probes and primers of the invention may be on the order of about 30 to 35 or so nucleotides in length. In some embodiments, the length of the selected oligonucleotide primer sequences (e.g., “forward” and “reverse” primers) and/or the length of the selected detection probe sequences (e.g., “anchor” and “sensor” probes), will likely be on the order of about 20 to 30 or so nucleotides in length, although in some cases, the sizes of particular probes and primer sequences may be larger than that, and on the order of about 60 to 70 nucleotides in length. Alternatively, in some embodiments, it may be desirable to employ shorter probe and/or primer sequences, and as such, the oligonucleotides selected for practice of the invention may be on the order of about 15 to 20 or so nucleotides in length or even slightly shorter in some embodiments.

In the context of the present application, it is understood that all intermediate oligonucleotide lengths within the various ranges stated herein are contemplated to expressly fall within the scope of the present invention. To that end, oligonucleotides that are less than about 60, less than about 59, less than about 58, less than about 57, less than about 56, etc. are expressly within the scope of the present disclosure, as are oligonucleotides that are less than about 50, less than about 49, less than about 48, less than about 47, less than about 46, etc., as well as oligonucleotides that are less than about less than about 40, less than about 39, less than about 38, less than about 37, less than about 36, etc. and so forth.

### 2.3.1 JAK2-Specific Amplification Primers

In the practice of the invention, forward and reverse amplification primers for use in the amplification of JAK2-specific polynucleotide sequences, and JAK2^{V617F}-encoding polynucleotide sequences in specifc, preferably comprise at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 or more contiguous nucleic acids from any one of the “forward” oligonucleotide primer sequences disclosed in SEQ ID NO:1, SEQ ID NO:2, SEQ ID NO:3, or SEQ ID NO:4 or the “reverse” oligonucleotide primer sequences disclosed in SEQ ID NO:5, SEQ ID NO:6, SEQ ID NO:7, SEQ ID NO:8, or SEQ ID NO:9; or from oligonucleotide sequences that are at least about 90% identical to any one of the “forward” oligonucleotide primer sequences disclosed in SEQ ID NO:1, SEQ ID NO:2, SEQ ID NO:3, or SEQ ID NO:4 or the “reverse” oligonucleotide primer sequences disclosed in SEQ ID NO:5, SEQ ID NO:6, SEQ ID NO:7, SEQ ID NO:8, or SEQ ID NO:9; or even from oligonucleotide sequences that are at least about 95% identical to any one of the “forward” oligonucleotide primer sequences disclosed in SEQ ID NO:1, SEQ ID NO:2, SEQ ID NO:3, or SEQ ID NO:4 or the “reverse” oligonucleotide primer sequences disclosed in SEQ ID NO:5, SEQ ID NO:6, SEQ ID NO:7, SEQ ID NO:8, or SEQ ID NO:9.

In other embodiments, the preferred oligonucleotide forward and reverse amplification primer sequences of the invention may comprise any one of the “forward” oligonucleotide primer sequences disclosed in SEQ ID NO:1, SEQ ID NO:2, SEQ ID NO:3, and SEQ ID NO:4 or the “reverse” oligonucleotide primer sequences disclosed in SEQ ID NO:5, SEQ ID NO:6, SEQ ID NO:7, SEQ ID NO:8, or SEQ ID NO:9, while in other embodiments, it may be desirable to employ primer sequences that consist essentially of any one of the “forward” oligonucleotide primer sequences disclosed in SEQ ID NO:1, SEQ ID NO:2, SEQ ID NO:3, and SEQ ID NO:4 or the “reverse” oligonucleotide primer sequences disclosed in SEQ ID NO:5, SEQ ID NO:6, SEQ ID NO:7, SEQ ID NO:8, or SEQ ID NO:9, and while in still other embodiments, it may be desirable to employ primer sequences that consist of any one of the “forward” oligonucleotide primer sequences disclosed in SEQ ID NO:1, SEQ ID NO:2, SEQ ID NO:3, and SEQ ID NO:4 or the “reverse” oligonucleotide primer sequences disclosed in SEQ ID NO:5, SEQ ID NO:6, SEQ ID NO:7, SEQ ID NO:8, or SEQ ID NO:9.

In yet additional embodiments, the forward and reverse amplification primer compositions preferred for the practice of the methods of the present invention may consist of a nucleic acid sequence that represents a contiguous nucleic acid sequence of about 6, about 7, about 8, about 9, about 10, about 11, about 12, about 13, about 14, about 15, about 16, about 17, about 18, about 19, or about 20 or more nucleotides as disclosed in any one of SEQ ID NO:I, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

Likewise, the primer compositions preferred for the practice of the amplification methods of the present invention may consist of a nucleic acid sequence that is about 90% identical to a contiguous nucleic acid sequence of about 6, about 7, about 8, about 9, about 10, about 11, about 12, about 13, about 14, about 15, about 16, about 17, about 18, about 19, or about 20, or more nucleotides as disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

Additionally, the amplification primer compositions preferred for the practice of the amplification methods of the present invention may consist of a nucleic acid sequence that is at least about 95% identical to any one of the oligonucleotide sequences disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

In other embodiments, the primer compositions preferred for the practice of the invention may consist essentially of a nucleic acid sequence that is at least about 6, at least about 7, at leat about 8, at least about 9, at least about 10, at least about 11, at least about 12, at least about 13, at least about 14, at least about 15, at least about 16, at least about 17, at least about 18, at least about 19, or at least about 20 or more contiguous nucleic acids selected from any one of the oligonucleotide sequences disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9; or may consist essentially of an oligonucleotide sequence that is at least about 90% identical to any one of the oligonucleotide sequences disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9; or even may consist essentially of an oligonucleotide sequence that is at least about 95% identical to any one of the oligonucleotide sequences disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

In certain embodiments, illustrative amplification primers of the invention are at least about 50 nucleotides in length and comprise, consist essentially or, or consist of a nucleotide sequence that comprises a nucleotide sequence as disclosed in any one of SEQ ID NO:1, SEQ ID NO:2, SEQ ID NO:3, SEQ ID NO:4, SEQ ID NO:5, SEQ ID NO:6, SEQ ID NO:7, SEQ IDNO:8, SEQ ID NO:9, or SEQ ID NO:10.

In other embodiments, illustrative amplification primers of the invention are at least about 40 nucleotides in length and comprise, consist essentially of, or consist of a nucleotide sequence that comprises a nucleotide sequence as disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In additional embodiments, illustrative amplification primers of the invention are at least about 30 nucleotides in length and comprise, consist essentially or, or consist of a nucleotide sequence that comprises a nucleotide sequence as disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In still additional aspects of the invention, illustrative amplification primers of the invention are at least about 25 nucleotides in length and comprise, consist essentially of, or consist of a nucleotide sequence that comprises a nucleotide sequence as disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, while in other aspects, illustrative amplification primers of the invention are at least about 20 or so nucleotides in length and comprise, consist essentially of, or consist of a nucleotide sequence that comprises a nucleotide sequence as disclosed in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In particular illustrative examples, the oligonucleotide primer compositions of the invention are less than about 50 nucleotides in length and comprise the nucleotide sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, while in other illustrative examples, the oligonucleotide primer compositions of the invention are less than about 40 nucleotides in length and comprise the nucleotide sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and in other exmples still, the oligonucleotide primer compositions of the invention are less than about 30 nucleotides in length and comprise the nucleotide sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In some aspects of the invention, it may be desirable to employ oligonucleotide primer compositions in the practice of the methods disclosed herein that are less than about 45 or so nucleotides in length and that consist essentially of the nucleotide sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or compositions that are less than about 35 or so nucleotides in length and that consist essentially of the nucleotide sequence of any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, while in other exmples still, it may be desirable to employ oligonucleotide primer compositions that are less than about 25 or so nucleotides in length and that consist essentially of the nucleotide sequence of any one of SEQ ID NO:1, SEQ ID NO:2, SEQ ID NO:3, SEQ ID.NO:4, SEQ ID NO:5, SEQ ID NO:6, SEQ ID NO:7, SEQ ID NO:8, SEQ ID NO:9, or SEQ ID NO:10.

### 2.3.2 JAK2-Specific Detection Probes

In the practice of the invention, anchor and sensor primers for use in the detection of JAK2-specific polynucleotide sequences, and JAK2^{V617F}-encoding-specific polynucleotide sequences in specifc, using the real-time PCR, FRET-based analyses described herein, will preferably comprise at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 or more contiguous nucleic acids from any one of the “anchor” oligonucleotide probe sequences disclosed in SEQ ID NO:10, SEQ ID NO:11, SEQ ID NO:12, SEQ ID NO:13, or SEQ ID NO:14, or the “sensor” oligonucleotide probe sequences disclosed in SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; or from oligonucleotide sequences that are at least about 90% identical to any one of the oligonucleotide detection probe sequences disclosed in SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; or even from oligonucleotide sequences that are at least about 95% identical to any one of the oligonucleotide probe sequences disclosed in SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In other embodiments, the preferred oligonucleotide detection probes of the invention may comprise any one of the oligonucleotide probe sequences disclosed in SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, while in other embodiments, it may be desirable to employ detection probe nucleic acid segments that consist essentially of any one of the oligonucleotides as disclosed in SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, and while in still other embodiments, it may be desirable to employ probe sequences that consist of any one of the oligonucleotide sequences disclosed in SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In yet additional embodiments, the detection probes of the present invention will preferably comprise a pair of probes, the first of which is an “anchor” probe, and the second of which is a “sensor” probe as described herein.

The probe compositions preferred for the practice of the methods of the present invention may comprise a pair of detection probes, the first and second members of which may consist of a nucleic acid sequence that represents a contiguous nucleic acid sequence of about 6, about 7, about 8, about 9, about 10, about 11, about 12, about 13, about 14, about 15, about 16, about 17, about 18, about 19, or about 20 or more nucleotides as disclosed in any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

Likewise, the probe compositions preferred for the practice of the detection methods of the present invention may comprise a pair of detection probes, the first member of which may consist of a nucleic acid sequence that is about 80%, at least about 81% identical, at least about 82% identical, at least about 83%, at leat about 84% or at least about 85% identical to a contiguous nucleic acid sequence of about 6, about 7, about 8, about 9, about 10, about 11, about 12, about 13, about 14, about 15, about 16, about 17, about 18, about 19, or about 20, or more nucleotides as disclosed in any one of SEQ ID NO:10, SEQ ID NO:11, SEQ ID NO:12, SEQ ID NO:13, or SEQ ID NO:14, while the second member of which pair of detection probes may preferably consist of a nucleic acid sequence that is about 90%, at least about 91% identical, at least about 92% identical, at least about 93%, at leat about 94% or at least about 95% identical to a contiguous nucleic acid sequence of about 6, about 7, about 8, about 9, about 10, about 11, about 12, about 13, about 14, about 15, about 16, about 17, about 18, about 19, or about 20, or more nucleotides as disclosed in any one of SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

Additionally, the probe compositions preferred for the practice of the methods of the present invention may comprise a pair of detection probes, the first and second members of which may comprise nucleic acid sequences that are at least about 95%, at least about 96% identical, at least about 97% identical, or at least about 98% or 99% identical to any one of the oligonucleotide sequences disclosed in any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In other embodiments, the probe compositions preferred for the practice of the invention may comprise a pair of detection probes, the first and second members of which may consist essentially of a nucleic acid sequence that is at least about 6, at least about 7, at leat about 8, at least about 9, at least about 10, at least about 11, at least about 12, at least about 13, at least about 14, at least about 15, at least about 16, at least about 17, at least about 18, at least about 19, or at least about 20 or more contiguous nucleic acids selected from any one of the oligonucleotide sequences disclosed in any one of SEQ ID NO:10, SEQ ID NO:11, SEQ ID NO:12, SEQ ID NO:13, SEQ IDNO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; or may consist essentially of an oligonucleotide sequence that is at least about 90% identical to any one of the oligonucleotide sequences disclosed in any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; or even may consist essentially of an oligonucleotide sequence that is at least about 95%, at least about 96% identical, at least about 97% identical, or at least about 98% or 99% identical to any one of the oligonucleotide sequences disclosed in any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In certain embodiments, illustrative detection probe compositions of the invention preferably are oligonucleotides of at least about 50 or so nucleotides in length, that either comprise, consist essentially of, or consist of, a nucleotide sequence selected from the group consisting of SEQ ID NO:10, SEQ ID NO:11, SEQ ID NO:12, SEQ ID NO:13, and SEQ ID NO:14; or that comprise, consist essentially of, or consist of, a nucleotide sequence selected from the group consisting of SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In other embodiments, illustrative detection probe compositions of the invention preferably are oligonucleotides of at least about 40 or so nucleotides in length, that either comprise, consist essentially of, or consist of, a nucleotide sequence selected from the group consisting of SEQ ID NO:10, SEQ ID NO:11, SEQ ID NO:12, SEQ ID NO:13, and SEQ ID NO:14; or that comprise, consist essentially of, or consist of, a nucleotide sequence selected from the group consisting of SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In other embodiments, detection probe compositions may be oligonucleotides of at least about 30 or so nucleotides in length, that either comprise, consist essentially of, or consist of, a nucleotide sequence selected from the group consisting of SEQ ID NO:10, SEQ ID NO:11, SEQ ID NO:12, SEQ ID NO:13, and SEQ ID NO:14; or that comprise, consist essentially of, or consist of, a nucleotide sequence selected from the group consisting of SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23, while in other embodiments still, suitable detection oligonucleotides may be at least about 20 or so nucleotides in length, that either comprise, consist essentially of, or consist of, a nucleotide sequence selected from the group consisting of SEQ ID NO:10, SEQ ID NO:11, SEQ ID NO:12, SEQ ID NO:13, and SEQ ID NO:14; or that comprise, consist essentially of, or consist of, a nucleotide sequence selected from the group consisting of SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In particular illustrative examples, the oligonucleotide detection probe compositions of the invention are less than about 50 or so nucleotides in length and comprise the nucleotide sequence of any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, while in other illustrative examples, the oligonucleotide detection probe compositions are less than about 40 nucleotides in length and comprise the nucleotide sequence of any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In other applications, suitable oligonucleotide detection probes may be less than about 30 nucleotides in length and may comprise a nucleotide sequence selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In some aspects of the invention, it may be desirable to employ oligonucleotide probe compositions in the practice of the methods disclosed herein that are less than about 45 or so nucleotides in length and that consist essentially of the nucleotide sequence of any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, or alternatively, oligonucleotide probes that are less than about 35 or so nucleotides in length and that consist essentially of the nucleotide sequence of any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23, while in other exmples still, it may be desirable to employ oligonucleotide detection probe compositions that are less than about 25 or so nucleotides in length and that consist essentially of the nucleotide sequence of any one of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

In illustrative embodiments, the invention provides JAK2-specific amplification primers and detection probes that comprise, consist essentially of, or consist of, nucleic acid sequences that are preferably at least about 80%, at least about 85%, at least about 90%, at least about 95%, at least about 96%, at least about 97%, at least about 98%, or at least about 99% or more identical to any one or more of the oligonucleotide sequences disclosed in SEQ ID NO: I through SEQ ID NO:23.

In related embodiments, the invention also provides JAK2^{V617F}-specific amplification primers and detection probes that comprise, consist essentially of, or consist of, nucleic acid sequences that are preferably at least about 80%, at least about 85%, at least about 90%, at least about 95%, at least about 96%, at least about 97%, at least about 98%, or at least about 99% or more identical to any one or more of the oligonucleotide sequences disclosed in SEQ ID NO:1 through SEQ ID NO:23.

The invention also provides JAK2^{V617F}-specific amplification primers and detection probes that comprise a nucleic acid sequence as disclosed in any one of SEQ ID NO:1 through SEQ ID NO:23, amplification primers and detection probes that consist essentially of a nucleic acid sequence as disclosed in any one of SEQ ID NO:1 through SEQ ID NO:23, and amplification primers and detection probes that consist of a nucleic acid sequence as disclosed in any one of SEQ ID NO:1 through SEQ ID NO:23.

In particular embodiments, the present invention provides sets of primers and probes designed based on their specific binding to one or more portions of a human JAK2 gene. The present invention also provides sets of primers and probes that are designed based upon a mutation in the human JAK2 gene which gives rise to translation of a mutated JAK2^{V617F} polypeptide. These probes and primers are particularly useful in a method for rapidly detecting and identifying mutated JAK2 polynucleotide sequences that encode a JAK2^{V617F} polypeptide in a biological sample. In particular embodiments, these methods involve real-time PCR FRET-based amplification, detection, and quantitation means.

The present invention provides sets of two amplification primers, which preferably comprise at least a first forward amplification primer, and at least a first reverse amplification primer. Exemplary JAK2^{V617F} forward amplification primers include those sequences as disclosed in SEQ ID NO:1 through SEQ ID NO:4, while exemplary JAK2^{V617F} reverse amplification primers include those sequences as disclosed in SEQ ID NO:5 through SEQ ID NO:9.

The present invention also provides sets of two detection primers, which preferably comprise at least a first anchor probe, and at least a first sensor probe. Exemplary JAK2^{V617F} anchor probes include those sequences as disclosed in SEQ ID NO:10 through SEQ ID NO:14, while exemplary JAK2^{V617F} sensor probes include those sequences as disclosed in SEQ ID NO:15 through SEQ ID NO:23.

A particularly preferred set of amplification primers include a first oligonucleotide that comprises the sequence of SEQ ID NO:1, and a second oligonucleotide that comprises the sequence of SEQ ID NO:5. A particularly preferred set of detection probes include a first oligonucleotide that comprises the sequence of SEQ ID NO:10, and a second oligonucleotide that comprises the sequence of SEQ ID NO:15. Illustrative examples employing these probes and primers in the practice of the invention are shown in section 4 hereinbelow.

### 2.4 Polynucleotide Amplicifcation Kits

The present invention also provides kits for amplifying mammalian DNA, and in particular, DNAs comprising one or more JAK2-encoding polynucleotides. Such kits typically comprise two or more components necessary for amplifying mammalian DNA, and such components may be compounds, reagents, containers and/or equipment. For example, one container within a kit may contain a first primer, while a second container within the kit may comprise a second primer. A third container within the kit may contain a set of hybridization probes, or one or more fluorescent probes for labeling the probes. In addition, the kits of the invention may also comprise instructions for use, e.g., instructions for using the primers in amplification and/or detection reactions as described herein, as well as one or more fluorescent molecules, or other reagents as may be necessary, including for example, but not limited to, buffers enzymes, polymerases, RNases and such like.

The invention provides one or more JAK2-specific oligonucleotide compositions together with one or more excipients, buffers carriers, diluents, adjuvants, and/or other components, as may be employed in the formulation of particular JAK2-specific oligonucleotide assay reagents, and in the preparation of diagnostic tools. In certain embodiments the invention provides amplification kits for the amplification of polynucleotides comprising the mutation which produces the JAK2^{V617F} mutation.

### 2.5 Kits for the Detection and Quantitation of JAK2^{V617F} Mutations

Diagnostic kits represent another aspect of the invention. Such kits may also comprise one or more distinct container means within the kit for the probes, primers, fluorescent labels, or reaction buffers, polymerases, etc. The kit may also further comprise instructions for using the compositions comprised within the kit in real-time PCR assays, and in real-time PCR FRET-based assays in particular. Instructions may also be provided for the use of the reagents contained within the kit for the detection of JAK2^{V617F} mutations in analysis of one or more biological fluids suspected of containing a plurality of polynucleotides that encode such a mutation.

The container means for such kits may typically comprise at least one vial, test tube, flask, bottle, syringe or other container means, into which the disclosed JAK2- or JAK2^{V617F}-specific oligonucleotide composition(s) may be placed, and preferably suitably aliquoted. Where a second JAK2 or JAK2^{V617F} primer composition is also provided, the kit may also contain a second distinct container means into which this second primer composition may be placed. Alternatively, the plurality of JAK2- or JAK2^{V617F}-specific oligonucleotide compositions may be prepared in a single formulation, and may be packaged in a single container means, such as a vial, flask, syringe, test tube, ampoule, or other suitable container means.

The various kits of the present invention will also typically include a means for containing the vial(s) contained therein in close confinement for commercial sale, such as, e.g., injection or blow-molded plastic containers, boxes, or other suitable commercial packaging, into which the desired vial(s) and/or reagent or kit components are retained. Likewise, the kits of the present invention also preferably comprise instructions for using the items contained within such kits in the real-time PCR based assays described herein, including, for example, the real-time PCR/microvolume fluorimetry FRET analyses facilitated by instrumentation such as the Roche LightCyclerO platform.

### 2.6 Real-Time PCR-Based Fret Detection

Real-time PCR and FRET methodologies have been well-described in the literature (see, for example, U.S. Pat. No. 4,996,143, U.S. Pat. No. 5,565,322, U.S. Pat. No. 5,849,489, and U.S. Pat. No. 6,162,603, each of which is specifically incorporated herein by reference in its entirety.) The LightCycler® platform represents a significant breakthrough in genetic mutation screening and analysis. This system incorporates a rapid, air-driven thermal cycling instrument that can perform 30 polymerase chain reaction (PCR) cycles in less than 20 minutes. It utilizes an in-line microvolume fluorirneter to detect and quantitate fluorescently-labeled hybridization probes, and provides the data necessary for determination of melting curve analyses. The LightCycler® platform provides innovative instrumentation to facilitate the development of genetic analysis tools, and to provide a rapid, qualitative method for the assay of specific nucleotide sequences, and genetic mutations. Detailed application of the instrumentation in amplification and detection methods may be found on the manufacturer's website, and in product application manuals. This technology has also been described, including for example PCT Intl. Appl. Publ. Nos. WO 97/46707, WO 97/46714 and WO 97/46712 (each of which is specifically incorporated herein by reference in its entirety).

A typical LightCycler® FRET probe system consists of a pair of single-stranded fluorescently-labeled oligonucleotides. The first probe (also called the “donor” probe) is labeled at its 3′-end with a donor fluorophore (e.g., generally fluorescein) and the second probe (also referred to as the “acceptor” probe) is labeled at its 5′-end with one of four commercially-available LightCycler fluorophores (e.g., Red 610, 640, 670 and 705). The free 3′ hydroxyl group of the second probe must be blocked with a phosphate group to prevent Taq DNA polymerase extension. To avoid any steric problems between the donor and the acceptor fluorophores on the two probes, probes are preferably designed where there is a spacer of between 1 to about 5 nt to separate the two probes from each other.

During the annealing step of real-time quantitative PCR, the PCR primers and the LightCycler® probes hybridize to their specific target regions causing the donor dye to come into close proximity to the acceptor dye. When the donor dye is excited by light from the LightCycler® instrument, energy is transferred from the donor to the acceptor dye (via FRET). This transfer of energy causes the acceptor dye to emit light at a longer wavelength than the light emitted from the LightCycler® instrument. This acceptor fluorophore's emission wavelength is then detected by the LightCycler® instrument's optical unit. The increase in measure fluorescent signal is directly proportional to the amount of accumulating target DNA.

### 2.7 Methods for Detection of the JAK2^{V617F} Mutation

The invention provides for methods of identifying mammalian JAK2 polynucleotide sequences, and in particular JAK2^{V617F}-encoding polynucleotide sequences in a sample. The invention also provides methods and compositions for specifically detecting JAK2 and JAK2^{V617F}-specific polynucleotide sequences in a sample, and particularly in a clinical or biological specimen obtained from a human.

The invention further provides methods for specifically detecting mutant, or non-wildtype human JAK2 polynucleotide sequences in a sample, and polynucleotides that comprose the JAK2^{V617F} mutant sequence, or nucleic acid segments that encode a mutated JAK2^{V617F} peptide or polypeptide. These methods preferably utilize the primer and probe compositions and kits disclosed herein for detecting PCR amplification products using a JAK2 target sequence, and particularly for detecting and quantitating such amplification products using FRET, and subsequent melting curve analysis.

In one embodiment, the invention provides a method for detecting the presence or absence of specific mutations in a JAK2 gene, JAK2 polynucleotide, or a nucleic acid segment that encodes all or a portion of a JAK2 peptide or polypeptide, and in particular the region corresponding to the area around codon 617 of such a JAK2 polypeptide. In certain aspects, one or more biological samples may be taken from an individual, and screened for the presence of such a sequence. In particular embodiments, the sample may be screened for the presence of one or more specific JAK2 mutations, including, for example, the JAK2^{V617F} mutation which has been implicated as relevant in a number of proliferative disorders.

In one aspect of the invention, there is provided a method for detecting the presence or absence of a JAK2 mutation, and in particular, a JAK2^{V617F} mutation in a plurality of polynucleotides comprised within a sample. In certain embodiments, the sample is a biological sample obtained from a mammal. Preferably the sample is obtained from a human being.

In particular application, these methods include a real-time PCR-based amplification step, which generally involves performing at least one cycling step (which includes at least a first “amplifying” step and at least a first “hybridizing” step). This amplifying step includes contacting the sample with a pair of JAK2- or JAK2^{V617F}-specific oligonucleotide primers to produce an amplification product if a JAK2 target polynucleotide was originally present in the sample. (If no target polynucleotide was originally present in the sample, then no specific product amplification would occur during the PCR process).

The hybridizing step typically includes contacting the sample that results from the amplifying step with a pair of JAK2-specific oligonucleotide probes. Generally, the first and second members of the pair of JAK2-specific oligonucleotide probes hybridizes to the amplification product within no more than about four or five nucleotides of each other. A first probe of the pair of detection probes is typically labeled with a donor fluorescent moiety and a second probe of the pair of detection probes is typically labeled with a corresponding acceptor fluorescent moiety. These detection probes, and the moieties that may be operably linked to them for use in the hybridizing step have been described in more detail hereinabove.

The method also further comprises at least the step of detecting the presence or absence of fluorescent resonance energy transfer (FRET) between the donor fluorescent moiety of the first probe and the acceptor fluorescent moiety of the second probe, wherein the presence of a FRET signal is usually indicative of the presence of the target polynucleotide in the sample. Conversely, the absence of a FRET signal is usually indicative of the absence of the polynucleotide in the population of nucleic acid sequences present in the sample.

The method also optionally further comprises an additional step of determining the melting temperature between one or both of the detection oligonucleotide probe(s) and the corresponding probe targets to confirm the presence or absence of the JAK2 specific sequence.

The process of determining the melting temperature between a probe and its corresponding target generally involve the following. The PCR products and the probes are cooled to the temperature around 40° C. to facilitate the probes to be annealed to the complimentary sequences of the targeted PCR products. Once the probes are anneal to the targeted PCR product, the fluorophobes on probes are brought to very closed positon allowing the FRET to occur and generate fluorence to be detected. Then, the temperature is increased very slowly at 0.1° C./second and the fluoresce intensity is monitored at a real-time fashion. As the temperature increases, some of the probles will begin to separate from the targeted product and the the fluorescence intensity decreases. With the real-time monitoring of the fluorescence, the temperature at which the fluorescence intestity decreases most dramatically can be determined using mathematic modeling. The tempearature is termed the melting temperature of the probes (Tₘ). The Tₘ is depending on the lenge of probes, the GC content of the probe and degree of complementary of sequences they are annealed to. Given the same probes, the completed complementary sequences will generate higher Tₘ than the mismatched sequences (With respect to the present invention, the former represents the wild type allele, while the latter repsents the mutant allele)

In one aspect, the detecting step includes exciting the biological sample at a wavelength absorbed by the donor fluorescent moiety and visualizing and/or measuring the wavelength emitted by the acceptor fluorescent moiety. In another aspect, the step of detecting can further comprise the step of quantitating the amount of FRET observed. In yet another aspect, the detecting step is performed after each cycling step, and further, can be performed in real-time.

The above-described methods can further include preventing amplification of a contaminant nucleic acid. Preventing amplification can include performing the amplifying step in the presence of uracil and treating the biological sample with uracil-DNA glycosylase prior to a first amplification step. In addition, the cycling step can be performed on a control sample. A control sample can include a portion of a nucleic acid molecule encoding a mammalian, and preferably, human, JAK2. Alternatively, such a control sample can be amplified using a pair of control primers and hybridized using a pair of control probes. The control primers and the control probes are usually other than the JAK2 primers and JAK2 probes, respectively. In such control reactions, a control amplification product is produced if a control template is present in the sample, and the control probes hybridize to the control amplification product.

In another aspect, the present invention provides a method for rapidly detecting in a biological sample, a mutated JAK2 polynucleotide sequence that encodes a JAK2^{V617F} polypeptide. In an overall and general sense, this method comprises amplification of a population of human DNAs suspected of containing such a mutation by polymerase chain reaction (PCR) using a specific primer set, hybridization of a specific probe set with the single-stranded PCR product, performing melting curve analysis and analyzing the Tₘ change of the hybrid of the single-stranded PCR product with the hybridization probes, thereby differentiating wild type JAK2-encoding DNAs from mutant DNAs which encode for a JAK2^{V617F} peptide or polypeptide.

In one embodiment, the present invention provides a method for rapidly detecting a polynucleotide that encodes a JAK2^{V617F} polypeptide, which comprises the steps of: (a) isolating DNA from a biological sample to be detected; (b) amplifying the DNA obtained from step (a) by polymerase chain reaction (PCR) using a primer set derived from the nucleotide sequences of a JAK2-encoding polynucleotide or a JAK2^{V617F}-encoding polynucleotide; (c) hybridizing a probe set based on the nucleotide sequences of the nucleotide sequences of a JAK2-encoding polynucleotide or a JAK2^{V617F}-encoding polynucleotide with the single-stranded PCR product obtained from step (b); and (d) performing melting curve analysis to analyze the Tm change of the hybrid of the single-stranded PCR product with the hybridization probes, thereby distinguishing wild type JAK2-encoding polynucleotides from mutant JAK2^{V617F}-encoding polynucleotides.

In step (c) the above described embodiment of the present invention, the probe set utilized in hybridization can specifically bind to the single-stranded PCR amplification product. Said probe set is used in combination with fluorescent labels, in which one probe is labeled with a fluorescent label and the other probe assists to produce fluorescence by fluorescence resonance energy transfer (FRET), whereby the PCR amplification product can be detected and quantified.

As described above, any label can be used as a label of an oligonucleotide probe as long as it fulfills the above-mentioned requirements and it interacts with a nucleic acid-specific label. Examples include, but are not limited to, LightCycler RED 640, LightCycler RED 705, TAMRA and Alexa Fluor 633. The label may be attached to an oligonucleotide at any position as long as the attachment does not influence the hybridization of the oligonucleotide. The label is preferably attached at the 5′ or 3′ end of the oligonucleotide.

Primers that amplify a JAK2 or JAK2^{V617F} polynucleotide may be designed using, for example, a computer program such as OLIGO (Molecular Biology Insights Inc., Cascade, Colo.). Typically, oligonucleotide primers are from about 7 or 8 to about 40 or 50 or so nucleotidesinlength (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 or so nucleotides in length). “JAK2 primers” as used herein refers to oligonucleotide primers that specifically anneal to nucleic acid sequences encoding a JAK2 polypeptide and initiate synthesis therefrom under appropriate conditions. Likewise, “JAK2^{V617F} primers” refers to oligonucleotide primers that specifically anneal to nucleic acid sequences that comprise the genetic mutation that encodes a JAK2^{V617F} mutation and initiate synthesis therefrom under appropriate conditions.

Designing oligonucleotides to be used as hybridization probes can be perfonned in a manner similar to the design of primers, although the members of a pair of probes preferably hybridize to an amplification product within no more than about 5 nucleotides of each other on the same strand such that fluorescent resonance energy transfer (FRET) can occur (e.g., within no more than 1, 2, 3, or 4 nucleotides of each other). This minimal degree of separation typically brings the respective fluorescent moieties into sufficient proximity such that FRET occurs. It is to be understood, however, that other separation distances (e.g., 6 or more nucleotides) are possible provided the fluorescent moieties are appropriately positioned relative to each other (for example, with a linker arm) such that FRET can occur.

The invention also concerns the use of one or more of the JAK2-specific oligonucleotide amplification primer or detection probe sets described herein in the detection of a mutation in a population of polynucleotides suspected of comprising at least a first polynucleotide that encodes a mammalian JAK2 polypeptide, and in particular, in the detection of a mutation in a population of polynucleotides suspected of comprising at least a first polynucleotide that encodes a mutant mammalian JAK2^{V617F} polypeptide.

### 2.8 Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more.”

Primer/Primer Sequence: A “primer” or “primer sequence” may include any nucleic acid sequence or segment that selectively hybridizes to a complementary template nucleic acid strand (“target sequence”) and functions as an initiation point for the addition of nucleotides to replicate the template strand. Primer sequences of the present invention may be labeled or contain other modifications which allow the detection and/or analysis of amplification products. In addition to serving as initiators for polymerase-mediated duplication of target DNA sequences, primer sequences may also be used for the reverse transcription of template RNAs into corresponding DNAs.

Sample: A “sample” as used herein means any sample containing nucleic acids, such as DNA or RNA. It may be a sample comprising a biological fluid, such as blood, serum, plasma, or cells, or tissues of an individual.

Structural gene: A polynucleotide, such as a gene, that is expressed to produce an encoded peptide, polypeptide, protein, ribozyme, catalytic RNA molecule, or antisense molecule.

Target Sequence: A “target sequence” or “target nucleotide sequence” as used herein includes any nucleotide sequence to which one of said primer sequences hybridizes under conditions which allow an enzyme having polymerase activity to elongate the primer sequence, and thereby replicate the complementary strand.

Transformation: A process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

Transformed cell: A host cell whose nucleic acid complement has been altered by the introduction of one or more exogenous oligo- or polynucleotides into that cell.

Vector: A nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

The terms “substantially corresponds to,” “substantially homologous,” or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more oligo- or polynucleotide sequences, the reference sequence will typically comprise at least about 10-15 nucleotides, more typically at least about 16 to 25 nucleotides, and even more typically at least about 26-35 nucleotides, at least about 40 nucleotides, at least about 45 nucleotides, at least about 50 nucleotides, or at least about 60, 70, 80, 90, or even at least about 100 or so nucleotides.

Preferably, when highly homologous fragments are desired, the percent identity between the two sequences (often referred to as “target” and “probe” sequences) will be at least about 80% identical, preferably at least about 85% identical, and more preferably at least about 90% identical, at least about 92% identical, at least about 93% identical, at least about 94% identical, or even at least about 95%, 96%, 97%, 98%, or 99% or higher. The percentage of homology or percentage of identity between 2 or more oligo- or polynucleotide sequences may readily be determined by one of skill in the art, using one or more of the standard sequence comparison algorithms, such as, e.g., the FASTA program analysis described by Pearson and Lipman (1988).

The term “naturally-occurring” as used herein as applied to an object refers to the fact that an object can be found in nature. For example, a polypeptide or polynucleotide sequence that is present in an organism that can be isolated from a source in nature and which has not been intentionally modified by the hand of man in a laboratory is naturally-occurring.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, 2, 3, 4, 5, 6, 7, 8, 9, or 10 base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary primer or probe sequences will typically bind quite specifically to the target sequence region of the plurality of polynucleotides and will therefore be highly efficient in directing amplification of the target sequence via real-time PCR.

Substantially complementary oligonucleotide sequences will be greater than about 80 percent complementary, greater than about 85 percent complementary, greater than about 90 percent complementary, or even greater than about 95 percent complementary (or “% exact-match”) to the corresponding target nucleic acid sequence to which the oligonucleotide specifically binds, and will, more preferably be greater than about 96% or higher complementary to the corresponding nucleic acid sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 96%, 97%, 98%, 99%, or even 100% complementary to all or a portion of the target nucleic acid sequence to which the designed oligonucleotide probe or primer specifically binds.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch¹⁹. Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov et al.²⁰, (2) a penalty of 3.0 for each gap and an additional 0.10 penalty for each symbol in each gap; and (3) no penalty for end gaps.

As used herein, “fluorescence resonance energy transfer pair” or “FRET pair” refers to a pair of fluorophores comprising a donor fluorophore and acceptor fluorophore, wherein the donor fluorophore is capable of transferring resonance energy to the acceptor fluorophore. In preferred fluorescence resonance energy transfer pairs, the absorption spectrum of the donor fluorophore does not substantially overlap the absorption spectrum of the acceptor fluorophore. As used herein, “a donor oligonucleotide probe” refers to an oligonucleotide that is labeled with a donor fluorophore of a fluorescent resonance energy transfer pair. As used herein, “an acceptor oligonucleotide probe” refers to an oligonucleotide that is labeled with an acceptor fluorophore of a fluorescent resonance energy transfer pair. As used herein, a “FRET oligonucleotide pair” will typically comprise an “anchor” or “donor” oligonucleotide probe and an “acceptor” or “sensor” oligonucleotide probe, and such a pair form a fluorescence resonance energy transfer (FRET) relationship when the donor oligonucleotide probe and the acceptor oligonucleotide probe are both hybridized to their complementary target nucleic acid sequences. Acceptable fluorophore pairs for use as fluorescent resonance energy transfer pairs are well known to those skilled in the art and include, but are not limited to, fluorescein/rhodamine, phycoerythrin/Cy7, fluorescein/Cy5, fluorescein/Cy5.5, fluorescein/LC Red 640, and fluorescein/LC Red 705.

### 3. BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1A, FIG. 1B, FIG. 1C, and FIG. 1D show representative JAK2 Real-Time PCR Melting Curves. Melting curves are drawn with dF/dT on the y-axis and temperature (° C.) on the x-axis. FIG. 1A: Melting curves from serial dilutions of the human erythroleukemia (HEL) cell line with the multiple myeloma (RPM16228) cell line. Dilutions include 1:0, 1:1, 1:2, 1:4, 1:10, 1:20 and 1:40. The RPMI8226 negative control and water blank are also shown. FIG. 1B: Melting curves from unstained bone marrow aspirate specimens. The HEL positive control, JAK2V617F positive case 2.06, JAK2V617F negative case 2.10, RPMI8226 negative control and water blank are shown. FIG. 1C: Melting curves from Wright's stained peripheral blood or bone marrow specimens. The HEL positive control, JAK2V617F positive case 3.09, JAK2V617F negative case 3.10, RPMI8226 negative control and water blank are shown. FIG. 1D: Melting curves from Wright's stained peripheral blood or bone marrow specimens. The HEL positive control, JAK2V617F positive case 3.08, JAK2V617F positive case 3.09, JAK2V617F negative case 3.10, RPMI8226 negative control and water blank are shown.

### 4. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 4.1 Example 1

### Rapid Detection of JAK2^{V617F} bu Melting Curve Analysis

This example describes a clinical assay method useful in the detection of the JAK2^{V617F} mutation in a population or plurality of nucleic acids. In these studies, specific amplification primers and FRET detection probes were designed, and patients with a biopsy proven diagnosis of a MPD or reactive condition were selected. DNA was extracted from fresh and archived peripheral blood and bone marrow specimens, and PCR melting curve analysis was performed on the LightCycler® platform. In about one hour's reaction time, the JAK2 region was successfully amplified; wild type amplicons demonstrated a melting temperature of approximately 54° C., whereas JAK2^{V617F} containing amplicons melted at approximately 45° C. Titration studies using cell lines with and without JAK2 mutations showed that the relative size of melting curve from wild and mutant alleles was proportional to ratios of cells with and without JAK2 mutation. The sensitivity of the assay allowed reliable detection of one homozygous mutant cell in 20 total cells. This mutation was identified in patients previously diagnosed with MPDs or acute myelogenous leukemia (AML) transformed from MPD, whereas a wild type genotype was identified in patients with reactive conditions or de novo AML.

### 4.1.1 Materials and Methods

Patient Samples and Cell Lines

Pathology and hematology reports at The Methodist Hospital (Houston, Tex.) were retrospectively reviewed to identify patients with diagnoses of primary myeloproliferative disorders, myelodysplastic syndromes, acute leukemia or reactive hematological disorders. Unused diagnostic materials were retrieved from the pathology storage facility, including twenty fresh peripheral blood specimens, twenty unstained peripheral blood or bone marrow aspirate smears, twenty Wright's stained peripheral blood or bone marrow aspirate smears and twenty unstained formalin fixed paraffin embedded clot sections. Human erythroleukemia (HEL) and multiple myeloma (RPMI8226) cell lines were used as positive and negative controls for the JAK2^{V617F} mutation.

DNA Extraction

DNA was extracted from the cell lines and patient samples using standard methods. Briefly, HEL or RPMI8226 cells were obtained from log-phase cultures, hematopoetic cells were scraped from glass slides or 100 μL of whole blood was transferred to a sterile 1.8 mL screw top microcentrifuge tube and digested with proteinase K at 55° C. for 120 min¹². DNA was then extracted using the DNeasy® Tissue Kit (Qiagen Inc., Valencia, Calif.) and eluted with 100 μL (50 μL twice) nuclease-free water. DNA concentration was spectrophotometrically measured by light absorption at 260 nm.

PCR Primers and Hybridization Probes

PCR primers were designed to flank the guanine to thymine transversion in codon 617 of the JAK2 gene, including forward primer JAKLCFP 5′-AAgCAgCAAgTATgATgAgCAA-3′ (Tₘ=66° C.) (SEQ ID NO:1) and reverse primer JAKLCRP 5′-AgCTgTgATCCTgAAACTgAA-3′ (Tₘ=65.6° C.) (SEQ ID NO:5). Fluorescence resonance energy transfer (FRET) probes were designed such that the 5′-probe overlapped the mutated codon and the 3′-probe annealed immediately downstream, including the “sensor” probe: LC-Red 5′-640-CAgA*C*ACATACTCCATAATTT-3′ (Tₘ=57.5° C.). (SEQ ID NO:10) and the “anchor” probe: LC-Fluorescein 5′-gTAgTTTTACTTACTCTCgTCTC-FITC-3′ (Tₘ=60.7° C.) (SEQ ID NO:15) where the asterisk denotes the nucleotide complementary to the wildtype/mutation site.

The human DNA sequence from clone RP11-39K24 on chromosome 9 contains the 3′ end of the JAK2 gene for Janus kinase 2 (a protein tyrosine is recorded at GenBank accession number AL161450). GenBank accession number NM_{—}004972 contains the human JAK2 polypeptide and polynucleotide sequences. The genetic mutation that gives rise to the JAK2^{V617F} mutated polypeptide occurs at nucleotide 55061 of this sequence, which corresponds to nucletotide 1849 in exon 12.

Real-Time PCR and Melting Curve Analysis

Codon 12 of the human JAK2 gene was amplified using the LightCycler platform (Roche Applied Diagnostics, Basel, Switzerland).

Shown here is the DNA sequence in the region of the mutation of the wild-type G at nucleotide 55061 (shown here underlined), and the corresponding areas to which the primer sequences and probe sequences selectively hybridize. In the case of the mutant, the wild-type guanosine at nucleotide 55061 (G) is replaced by thymine (T). This single nucleotide change gives rise to the valine to phenylalanine mutation when mRNA transcribed from the region is translated into its amino acid product:

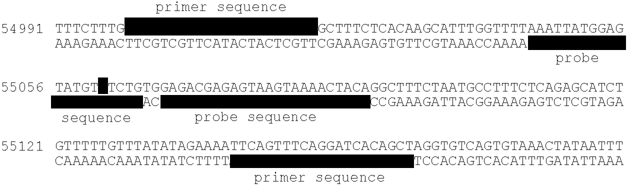

The regions where the primer sequences preferably bind are shown in bold, while the regions of the DNA where the detection probe sequences preferably bind are shown in italics.

Real-time PCR was performed on each specimen using either 5.0 μL of purified genomic DNA extract in a total reaction volume of 20 μL that included 4 μL of FastStart DNA Master^{PLUS TM} SYBR Green I 5× reaction master mix (Roche Applied Diagnostics, Basel, Switzerland), 2.0 μL JAK2LCFP (5 μM), 2.0 μL JAK2LCRP(5 μM), 1.0 μL LCFN (10 μM), 1.0 μL LCRD (10 μM) and 5.0 μL nuclease free water. PCR cycle parameters were an initial denaturing step consisting of 1 cycle at 95° C. for 10 min followed by an amplification step consisting of 55 cycles of denaturing at 95° C. for 10 sec, annealing at 60° C. for 60 sec and amplifying at 75° C. for 10 sec. LightCycler® System DNA melting curve analysis was performed by denaturing at 95° C. for 10 sec, annealing at 29° C. for 60 sec and melting by a transition rate of 0.20° C./sec to 70° C. Melting curves were visually analyzed and the Tₘ of each sample was electronically recorded.

Agarose Gel Analysis and DNA Sequencing

Amplification was verified by visualization of an appropriately-sized 163-basepair band when 5 μL of the real-time PCR product was applied to a 2% agarose gel and subjected to electrophoresis at 125 mV for 10 min. The JAK2 mutation determined by the above assay was verified by sequencing initially. Real-time PCR amplicons generated in the absence of hybridization probes were purified using a QlAquick PCR purification kit (QIAGEN Inc., Valencia, Calif.) according to the manufacturer's instructions. The sequencing reaction was then performed using the DTCS Quick Start™ Kit (Beckman Coulter Inc., Fullerton, Calif.) according the manufacturer's instructions. 1 μL of each purified amplicon was sequenced on a Peltier Thermal Cycler 200 (MJ Research Inc./BioRad Laboratories Inc., San Francisco, Calif.) with the JAK2LCFP forward primer or JAK2LCRP reverse primer through 30 cycles consisting of a denaturation step at 96° C. for 20 sec, an annealing step at 50° C. for 20 sec and an extension step at 60° C. for 120 sec. The resulting product was resolved by capillary electrophoresis on the CEQ8000 Genetic Analysis System (Beckman Coulter Inc., Fullerton, Calif.) according to the manufacturer's instructions, and the electropherogram was visually inspected to identify the presence of the wildtype or mutant genotype at codon 617.

### 4.1.2 Results

Optimization of PCR Amplification and DNA Melting Curve Analysis

The JAK2 LightCycler assay was optimized for real-time PCR amplification and melting curve analysis. Using a standard reaction mix, 100 ng of purified genomic DNA extract from either the HEL human erythroleukemia cell line, the RPMI8226 multiple myeloma cell line or a 1:1 mixture of each was amplified for 30, 40, 55 and 70 cycles. The ability to visualize the Tₘ inflection point of melted DNA was enhanced by plotting the negative first derivative (dF/dT) versus T. The dF/dT peak height ratio was optimal at 55 cycles, and additional cycles did not significantly improve this ratio. Similarly, the optimal starting temperature for melting curve analysis was determined by varying the temperature starting from 25° C. to 36° C. A 29° C. starting temperature was shown to generate optimal results.

Precision ofLightCycler System Melting Curve Analysis

The precision of the JAK2 LightCycler assay was determined by repeating the real-time PCR amplification and DNA melting curve analysis ten separate times using HEL and RPMI8226 genomic DNA. The JAK2 wildtype amplicon had a mean Tₘ equal to 54.28° C. and a coefficient of variation (CV) of 0.42% (Table 1). The JAK2^{V617F} mutant amplicon had a mean Tₘ equal to 45.48° C. and a coefficient of variation (CV) of 0.44% (Table 1). These statistical results are comparable to similar studies¹³.

TABLE 1
PRECISION AND REPRODUCIBILITY OF THE JAK2
REAL-TIME PCR MELTING-CURVE ASSAY

| | |
|---|---|
| Precision | Reproducibility |

| HEL | RPM18226 | HEL | RPM18226 |
|---|---|---|---|
| 45.52 | 54.41 | 45.84 | 54.53 |
| 45.57 | 54.49 | 45.90 | 54.33 |
| 45.52 | 54.07 | 45.85 | 54.41 |
| 45.48 | 54.35 | 46.00 | 54.35 |
| 45.65 | 54.26 | 45.70 | 54.41 |
| 45.06 | 54.37 | 45.89 | 54.34 |
| 45.24 | 53.96 | 45.91 | 54.15 |
| 45.46 | 54.41 | 45.71 | 54.27 |
| 45.52 | 53.91 | 45.83 | 54.11 |
| 45.78 | 54.59 | 45.88 | 54.32 |
| x: 45.48 | x: 54.28 | x: 45.85 | x: 54.32 |
| cv: 0.44 | cv: 0.42 | sd: 0.09 | sd: 0.12 |

Melting temperatures (° C.) for the human erythroleukernia (HEL) and multiple myeloma (RPMI8226) cell lines are shown. Average Tₘ (x), standard deviation (sd) and coefficient of variation (cv) are listed below.

Reproducibility ofLightCycler System Melting Curve Analysis

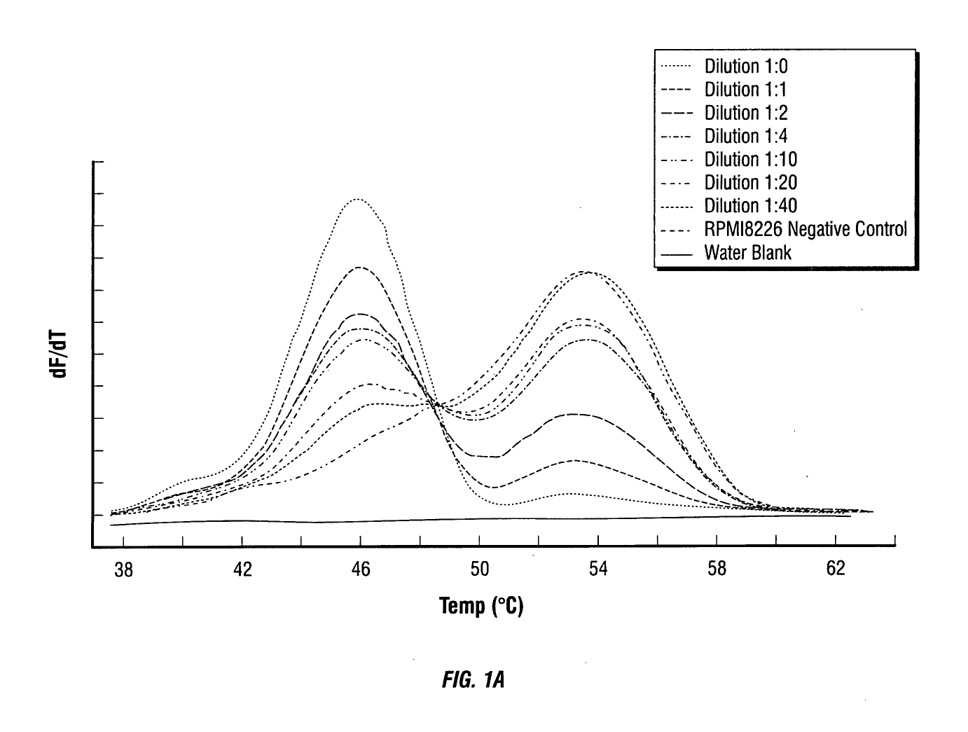

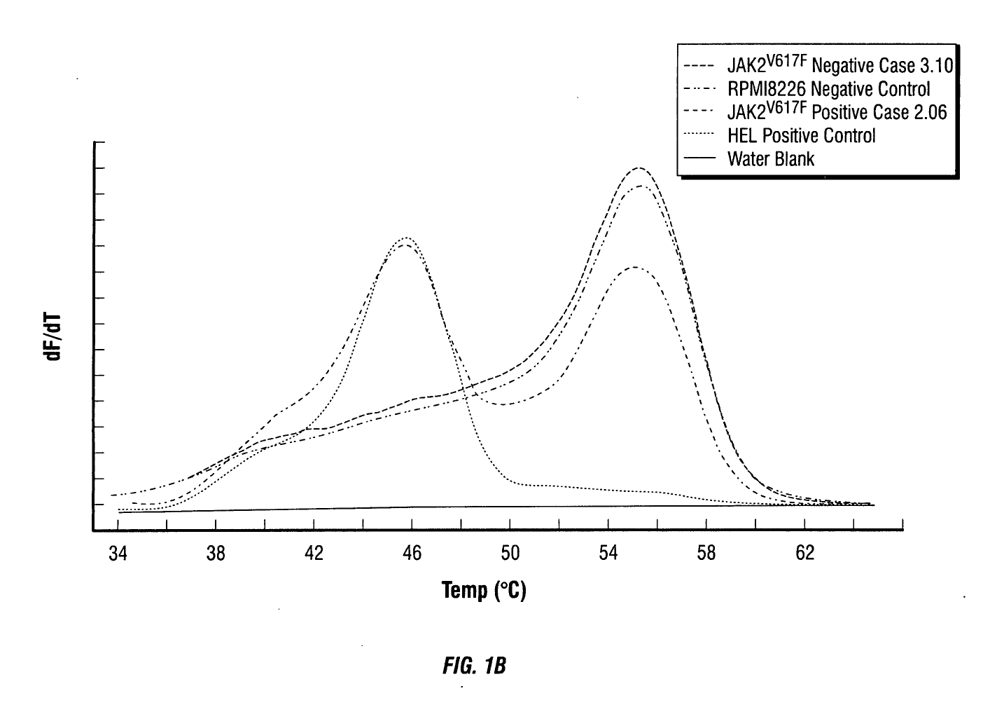

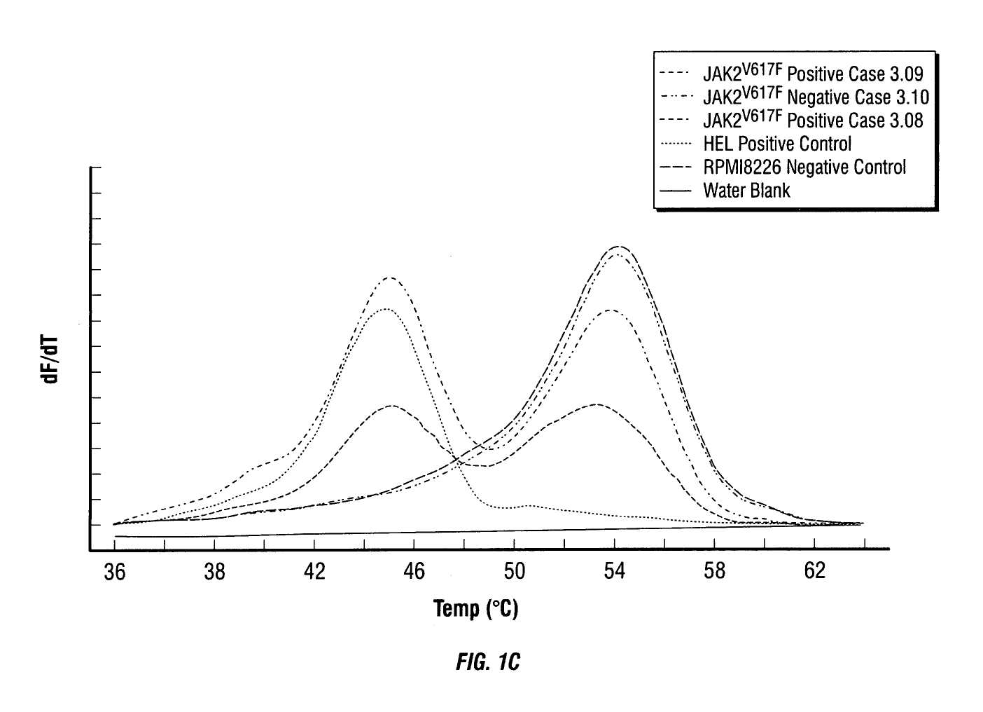

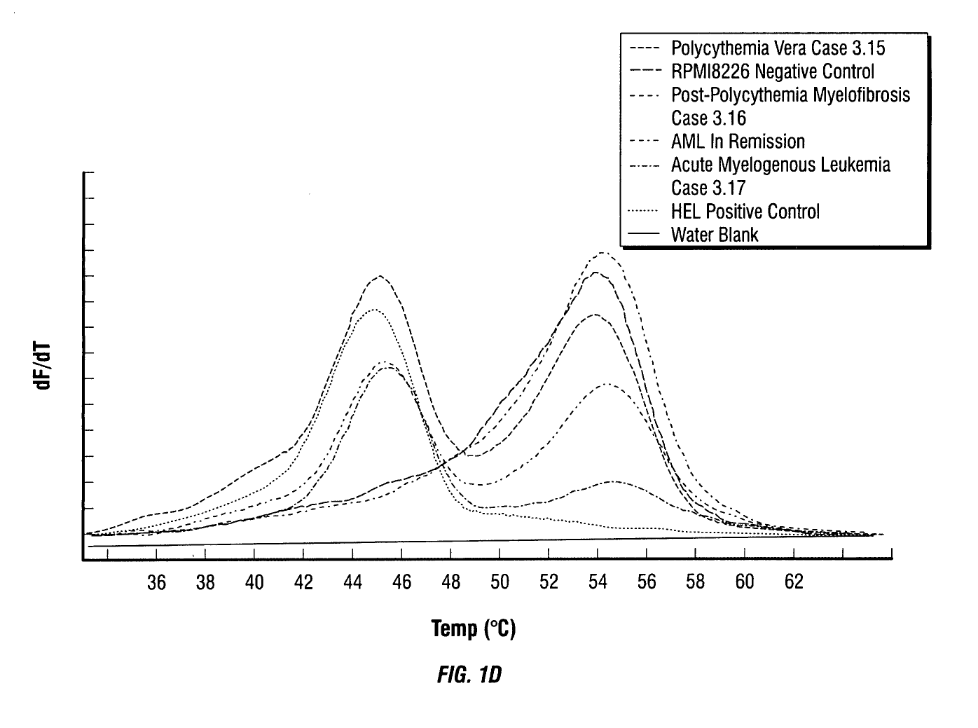

The reproducibility of the JAK2 LightCycler assay was determined by repetitive real-time PCR amplification and melting curve analysis of the HEL and RPMI8226 genomic DNA. Reproducible dF/dT versus T curves were generated. The mean Tₘ and standard deviation (SD) of the wildtype JAK2 amplicon was 54.32° C.±0.12, and the mean and SD of the JAK₂^{V617F} mutant amplicon was 45.85° C±0.09 (FIG. 1A; Table 1).

Lower Limitfor Detecting thre JAK2 Codon 617 Mutation

The ability of the JAK2 LightCycler assay to identify low concentrations of the V617F allele was evaluated by titration of the mutant HEL cell line genomic DNA with increasing concentrations of the wildtype RPMI8226 cell line genome. Serial dilutions of HEL:RPMI8226 of 1:1, 1:2, 1:5, 1:10, 1:20 and 1:40 were amplified by real-time PCR and analyzed by melting curve analysis. The two respective melting curves were easily distinguishable from the 1:1 to 1:20 dilutions; however, the mutant allele curve was inconsistently detected at the 1:40 dilution (FIG. 1B). Thus, the lower limit of detection for JAK2^{V617F} was defined as one copy per twenty alleles. The relative area under the mutant curve was inversely proportional to the area under the wildtype curve as the mutant to wildtype dilution factor increased (FIG. 1B).

TABLE 2
JAK2 REAL-TIME PCR MELTING CURVE ANALYSIS FROM
PERIPHERAL BLOOD SPECIMENS

| | | WT | V617FT | |
|---|---|---|---|---|
| Case | HemePath | Tₘ | Tₘ | JAK2 |
| # | Diagnosis | (° C.) | (° C.) | Mutation |
| 1.01 | Normal | 54.41 | — | Negative |
| 1.02 | Normal | 54.24 | — | Negative |
| 1.03 | Normal | 54.13 | — | Negative |
| 1.04 | Normal | 54.47 | — | Negative |
| 1.05 | Normal | 54.24 | — | Negative |
| 1.06 | Normal | 54.54 | — | Negative |
| 1.07 | Normal | 54.69 | — | Negative |
| 1.08 | Normal | 54.58 | — | Negative |
| 1.09 | Normal | 54.11 | — | Negative |
| 1.10 | Normal | 54.24 | — | Negative |
| 1.11 | Normal | 54.24 | — | Negative |
| 1.12 | Normal | 54.07 | — | Negative |
| 1.13 | Normal | 54.12 | — | Negative |
| 1.14 | Normal | 54.54 | — | Negative |
| 1.15 | Normal | 54.42 | — | Negative |
| 1.16 | Normal | 54.18 | — | Negative |
| 1.17 | Normal | 54.48 | — | Negative |
| 1.18 | Normal | 54.11 | — | Negative |
| 1.19 | Normal | 54.08 | — | Negative |
| 1.20 | Normal | 54.14 | — | Negative |

Melting temperatures (° C.) for the JAK2 wildtype (WT) and mutant (V6 17F) alleles are shown. Presence or absence of the mutant allele was interpreted as (positive) or (negative), respectively.

JAK2^{V617F} Identification in Myeloproliferative Disorders and Reactive Conditions

The JAK2 LightCycler assay was further challenged with twenty fresh peripheral blood specimens, twenty unstained bone marrow aspirate smears, twenty Wright's stained peripheral blood or bone marrow aspirate smears and twenty paraffin-embedded formalin-fixed bone marrow clot sections from patients previously diagnosed with a myeloproliferative disorder or other reactive condition. The wildtype allele was identified in each fresh peripheral blood specimen (mean Tₘ 54.30° C.±0.20) (Table 2, cases 1.01-1.20), unstained smear (mean Tₘ of 54.51° C.±0.55) (Table 3, cases 2.01-2.20) and Wright's stained smear (mean Tₘ of 54.16° C.±0.53) (Table 4, cases 3.01-3.20).

TABLE 3
JAK2 REAL-TIME PCR MELTING CURVE ANALYSIS FROM
UNSTAINED BONE MARROW ASPIRATE SPECIMENS

| | | WT | V617FT | |
|---|---|---|---|---|
| Case | HemePath | Tₘ | Tₘ | JAK2 |
| # | Diagnosis | (° C.) | (° C.) | Mutation |
| 2.01 | ET | 54.57 | — | Negative |
| 2.02 | Fib, Inc Bl | 54.96 | — | Negative |
| 2.03 | AML | 54.61 | — | Negative |
| 2.04 | Remission | 55.77 | — | Negative |
| 2.05 | MDS-NOS | 55.13 | — | Negative |
| 2.06 | MF | 54.43 | 45.41 | Positive |
| 2.07 | B-CLL | 53.13 | — | Negative |
| 2.08 | MDS-NOS | 53.45 | — | Negative |
| 2.09 | MDS-RARS | 54.48 | — | Negative |
| 2.10 | Normal | 54.48 | — | Negative |
| 2.11 | MF | 54.74 | — | Negative |
| 2.12 | AML post MF | 54.64 | 45.48 | Postive |
| 2.13 | Remission | 54.83 | — | Negative |
| 2.14 | Remission | 54.82 | — | Negative |
| 2.15 | MF | 54.39 | 45.42 | Positive |
| 2.16 | Normal | 54.47 | — | Negative |
| 2.17 | MDS-RCMD | 54.39 | — | Negative |
| 2.18 | MDS-NOS | 54.06 | — | Negative |
| 2.19 | MDS-RAEB | 54.35 | — | Negative |
| 2.20 | ME post PV | 54.55 | 45.39 | Positive |
| Melting temperatures (° C.) for the JAK2 wildtype (WT) and mutant (V6 17F) alleles are shown. Presence or absence of the mutant allele was interpreted as (positive) or (negative), respectively. The hematopathological diagnoses included acute myelogenous leukemia (AML), B-cell chronic lymphocytic leukemia (B-CLL), essential thrombocytosis (ET), nonspecific fibrosis with increased blasts (Fib, Inc Bl), myelodysplastic syndrome not otherwise specified (MDS-NOS), | | | | |
| # refractory anemia with excess blasts (MDS-RAEB), refractory anemia with ringed sideroblasts (MDS-RARS), refractory cytopenia with multilineage dysplasia (MDS-RCMD), myelofibrosis (MF), myelofibrosis following polycythemia vera (ME post PV) and acute leukemia in remission (Remission). | | | | |

TABLE 4
JAK2 REAL-TIME PCR MELTING CURVE ANALYSIS FROM
WRIGHT'S-STAINED PERIPHERAL BLOOD AND BONE
MARROW ASPIRATE SPECIMENS

| | | WT | V617FT | |
|---|---|---|---|---|
| Case | HemePath | Tₘ | Tₘ | JAK2 |
| # | Diagnosis | (° C.) | (° C.) | Mutation |
| 3.01 | MDS-NOS | 53.81 | — | Negative |
| 3.02 | Remission | 54.11 | — | Negative |
| 3.03 | Fib | 53.46 | — | Negative |
| 3.04 | Fib | 55.12 | — | Negative |
| 3.05 | MDS-RCMD | 53.01 | — | Negative |
| 3.06 | MDS-RAEB | 54.41 | — | Negative |
| 3.07 | Normal | 54.76 | — | Negative |
| 3.08 | AML post MF | 53.65 | 45.13 | Positive |
| 3.09 | ET | 53.65 | 45.33 | Positive |
| 3.10 | MDS-NOS | 54.97 | — | Negative |
| 3.11 | MDS-NOS | 54.03 | — | Negative |
| 3.12 | MDS-RCMD | 54.02 | — | Negative |
| 3.13 | MDS-RCMD | 54.03 | — | Negative |
| 3.14 | MDS-RCMD | 54.02 | — | Negative |
| 3.15 | PV | 53.91 | 45.03 | Positive |
| 3.16 | MF post PV | 54.21 | 45.29 | Positive |
| 3.17 | AML post MF | 54.41 | 45.29 | Positive |
| 3.18 | Remission | 54.12 | — | Negative |
| 3.19 | Remission | 54.49 | — | Negative |
| 3.20 | Remission | 54.92 | — | Negative |
| Melting temperatures (° C.) for the JAK2 wildtype (WT) and mutant (V617F) alleles are shown. Presence or absence of the mutant allele was interpreted as (positive) or (negative), respectively. The hematopathological diagnoses included acute myelogenous leukemia post myelofibrosis (AML post MF), essential thrombocytosis (ET), nonspecific fibrosis (Fib), myelodysplastic syndrome not otherwise specified (MDS-NOS), refractory cytopenia with multilineage dysplasia (MDS-RCMD), | | | | |
| # myelofibrosis following polycythemia vera (MF post PV), polycythemia vera (PV) and acute leukemia in remission (Remission). | | | | |

The wildtype allele was identified in only one half (50%) of the processed clot sections (mean Tₘ 54.50° C.±0.37) (Table 5, cases 4.01-4.20). The JAK2^{V617F} mutant allele was identified in none of the peripheral blood specimens (Table 2), four of the unstained smears (mean Tₘ of 45.43° C.±0.04) (examples shown in FIG. 1C; Table 3, cases 2.06, 2.12, 2.15 and 2.20), five of the stained smears (mean Tₘ of 45.21° C.±0.13) (examples shown in FIG. 1D; Table 4, cases 3.08-3.09 and 3.15-3.17) and nine of the clot sections (mean Tₘ of 45.37° C.±0.15) (Table 5, cases 4.01-4.03, 4.10-4.11, 4.13-4.15 and 4.20). These JAK2^{V617F} positive cases represented patients with polycythemia vera (PV), essential thrombocytosis (ET), idiopathic myelofibrosis (MF) and leukemia transformed from a preexisting MPD (1/1). Of note, concordant presence or absence of the JAK2^{V617F} mutant allele was observed in three patients having multiple specimens and/or specimen types (Table 6).

TABLE 5
JAK2 REAL-TIME PCR MELTING CURVE ANALYSIS FROM
FORMALIN FIXED PARAFFIN EMBEDDED BONE MARROW
CLOT SPECIMENS

| | Heme | WT Tₘ | V617F | |
|---|---|---|---|---|
| Case # | Diagnosis | (° C.) | Tₘ (° C.) | JAK2 Mutation |
| 4.01 | MF | 54.51 | 45.39 | Positive |
| 4.02 | CMML | 54.15 | 45.59 | Positive |
| 4.03 | CMML | 54.50 | 45.39 | Positive |
| 4.04 | MDS-NOS | — | — | Unknown |
| 4.05 | MDS-RCMD | — | — | Unknown |
| 4.06 | Fib | — | — | Unknown |
| 4.07 | Normal | — | — | Unknown |
| 4.08 | MF | — | — | Unknown |
| 4.09 | Myeloma | 54.71 | — | Negative |
| 4.10 | MDS-NOS | 54.87 | 45.69 | Positive |
| 4.11 | MF | 54.00 | 45.59 | Positive |
| 4.12 | Remission | — | — | Unknown |
| 4.13 | ET | 54.71 | 45.39 | Positive |
| 4.14 | MF post PV | 54.81 | 45.44 | Positive |
| 4.15 | ME | 55.01 | 45.34 | Positive |
| 4.16 | ET | — | — | Unknown |
| 4.17 | ET | — | — | Unknown |
| 4.18 | PV | — | — | Unknown |
| 4.19 | Fib | — | — | Unknown |
| 4.20 | MF | 54.71 | 44.53 | Positive |
| Melting temperatures (° C.) for the JAK2 wildtype (WT) and mutant (V617F) alleles are shown. Presence or absence of the mutant allele was interpreted as (positive) or (negative), respectively. The hematopathological diagnoses included chronic myelomonocytic leukemia (CMML), essential thrombocytosis (ET), nonspecific fibrosis (Fib), myelodysplastic syndrome not otherwise specified (MDS-NOS), refractory cytopenia with multilineage dysplasia (MDS-RCMD), myelofibrosis following | | | | |
| # polycythemia vera (MF post PV), myelofibrosis (MF), multiple myeloma (myeloma), polycythemia vera (PV) and acute leukemia in remission (Remission). | | | | |

TABLE 6
JAK2 REAL-TIME PCR MELTING CURVE ANALYSIS FROM
PATIENTS HAVING MULTIPLE SPECIMEN AND/OR
SPECIMEN TYPES

| Case | | Time | HemePath | JAK2 |
|---|---|---|---|---|
| # | Type | (months) | Diagnosis | V617F |
| 2.06 | PB | 0 | MF | Positive |
| 4.01 | Clot | 0 | MF | Positive |
| 2.17 | PB | 0 | MDS-RCMD | Negative |
| 3.05 | Asp | 0 | MDS-RCMD | Negative |
| 3.15 | Asp | 0 | PV | Positive |
| 3.16 | Asp | 1 | ME post PV | Positive |
| 4.14 | Clot | 1 | MF post PV | Positive |
| 2.12 | PB | 3 | AML post MF | Postive |
| 3.17 | Asp | 3 | AML post MF | Positive |
| 2.13 | PB | 4 | Remission | Negative |
| 2.14 | PB | 5 | Remission | Negative |
| 3.18 | Asp | 4 | Remission | Negative |
| 3.19 | Asp | 5 | Remission | Negative |
| 3.20 | Asp | 6 | Remission | Negative |
| Melting temperatures (° C.) for the JAK2 wildtype (WT) and mutant (V617F) alleles are shown. Specimen types included bone marrow aspirate (Asp), bone marrow clot section (Clot) and peripheral blood (PB). Time (months) from the first specimen is shown. Hematopathological diagnosis included acute myelogenous leukemia post myelofibrosis (AML post MF), refractory cytopenia with multilineage dysplasia (MDS-RCMD), myelofibrosis following polycythemia vera (MF post PV), myelofibrosis (MF), | | | | |
| # polycythemia vera (PV) and acute leukemia in remission (Remission). | | | | |

The present invention provides the first real-time PCR assay for the identification of JAK2^{V617F} using FRET detection and DNA melting-curve analysis. This assay precisely and reproducibly identified the presence or absence of the mutation in fresh and archived peripheral blood and bone marrow specimens (FIG. 1A-FIG. 1D; Table 2, Table 3, Table 4, and Table 5). The sensitivity of this assay was determined to be 1 mutant copy per 20 alleles (i.e., 5% mutant alleles). This sensitivity is compatible to that of pyro-sequencing and is more sensitive than conventional sequencing methods reported as far. Furthermore, this assay provides semiquantitative allelic frequency estimates as shown in FIG. 1A.

This assay provides an ideal clinical diagnostic assay for screening the JAK2^{V617F} mutation in patients who present with symptoms suggestive of MPDs other than chronic myeloid leukemia. The vast majority of patients tested will not have severe lymphocytosis which may potentially dilute the granunocytes carrying mutant alleles. This assay's sensitivity of detecting one JAK2^{V617F} mutant allele in total 20 alleles allows the elimination of procedures needed for sorting the granulocytic cells from non-neoplastic lymphocytes while using conventional sequencing methods. These procedures require magnetic beads or flow cytometry sorters are too costly and too time/labor demanding for clinical practice. A negative test will indicate the patients do not have significant amount of mutant alleles and thus are at very low risk of having MPDs associated with this mutation. Additionally, the assay described herein can be completed in only about 90 min after DNA preparation, thus generating results in a time-frame that allows reasonable turn-around-time to diminish the anxiety of patients waiting for test results.

Another major advantage of this assay is the ability to use various archived specimens providing the possibility of performing retrospective studies. This together with the ability of semiquantitative allelic frequency estimation may allow us to retrospectively investigate the role of JAK2^{V617F} mutation in disease progression or treatment response. As a pilot example, in the current study, multiple specimens from various time points were available for one patient having a long history of polycythemia vera that progressed to myelofibrosis and eventually transformed to acute myelogenous leukemia (Table 6). Comparison of the melting curves demonstrated this patient to have an increasing JAK2^{V617F} to wildtype proportion during disease progression, whereas the mutant allele was no longer detectable following allogenic bone marrow transplant (FIG. 1A-FIG. 1D; Table 6). Using a large retrospective study, it may also be possible to determine the disease progression at the molecular level prior to its clinical manifestation leading to potentially earlier therapeutic intervention. Similarly, a lack of JAK2^{V617F} allele following treatment may confirm the efficacy of treatment following conventional therapy or rationally designed therapeutics such as small molecule tyrosine kinase inhibitors.

In summary, the currently described real-time PCR assay with melting curve analysis represents a suitable clinical molecular diagnostic assay for detecting JAK2^{V617F} mutation with the characteristics of simple sample processing, good turn-round-time and excellent precision, reproducibility and sensitivity. Furthermore, this assay permits the use of archived materials allowing large scale retrospective studies to determine the significance JAK2^{V617F} mutation in disease progression and prognosis in the patients carrying this mutation.

### 5.0 EXEMPLARY SEQUENCES

In addition to the specific primer and probe sets described in illustrative embodiments above, the inventor also contemplates the use of additional primer and probe sets in the methods disclosed herein. In particular, the following forward and reverse primer oligonucleotide sequences (Table 7), and the following donor and acceptor oligonucleotide probe sequences are also intended for use in the practice of the disclosed methods. The probe sequences may be labeled with suitable donor and acceptor fluorophores as described in the examples herein.

TABLE 7
EXEMPLARY JAK2 PRIMER AND PROBE SEQUENCES

| | | | |
|---|---|---|---|
| | SEQ | | SEQ |
| | ID | | ID |
| Forward Primer | NO: | Reverse Primer | NO: |

PRIMERS:

| AAGCAGCAAGTATGATGAGCAA | 1 | AGCTGTGATCCTGAAACTGAA | 5 |
|---|---|---|---|
| AAGCAGCAAGTATGATGAG | 2 | GTGATCCTGAAACTGAATTTTCT | 6 |
| AGCAGCAAGTATGATGAG | 3 | GTGATCCTGAAACTGAATTTTCTA | 7 |
| GAAGCAGCAAGTATGATGAG | 4 | GTGATCCTGAAACTGAATTTTCTAT | 8 |
| | | GTGATCCTGAACTGAATTTTCT | 9 |
| | SEQ | | SEQ |
| | ID | | ID |
| Anchor Probe | NO: | Sensor Probe | NO: |

PROBES:

| | | | |
|---|---|---|---|
| AGCTGTGATCCTGAAACTGAA | 10 | GTAGTTTTACTTACTCTCGTCTC | 15 |
| AAAGGCATTAGAAAGCCTGTAGTTTTACTTACTC | 11 | CGTCTCCACAGACACATACTCC | 16 |
| AGAAAGGCATTAGAAAGCCTGTAGTTTTACT | 12 | CGTCTCCACAGACACATACTCCA | 17 |
| AGGCATTAGAAAGCCTGTAGTTTTACTTACT | 13 | CGTCTCCACAGACACATACTCCATA | 18 |
| CTGAGAAAGGCATTAGAAAGCCTGTAGTTTTACTTA | 14 | CGTCTCCACAGACACATACTCCATAA | 19 |
| | | CTCGTCTCCACAGACACATACT | 20 |
| | | CTCTCGTCTCCACAGACACATAC | 21 |
| | | GTCTCCACAGACACATACTCCATAAT | 22 |
| | | GTCTCCACAGACACATACTCCATAATT | 23 |

### 6.0 REFERENCES

The following references, to the extent that they provide exemplary procedural or other details supplementary to those set forth herein, are specifically incorporated herein by reference.
^{1.} E. J. Baxter, L. M. Scott, P. J. Campbell, C. East, N. Fourouclas, S. Swanton, G. S. Vassiliou, A. J. Bench, E. M. Boyd, N. Curtin, M. A. Scott, W. N. Erber and A. R. Green; “Acquired mutation of the tyrosine kinase JAK2 in human myeloproliferative disorders,” Lancet, 365:1054-1061, 2005.
^{2.} C. James, V. Ugo, J. P. Le Couedic, J. Staerk, F. Delhommeau, C. Lacout, L. Garcon, H. Raslova, R. Berger, A. Bennaceur-Griscelli, J. L. Villeval, S. N. Constantinescu, N. Casadevall and W. Vainchenker: A unique clonal JAK2 mutation leading to constitutive signalling causes polycythaemia vera, Nature, 434:1144-1148, 2005.
^{3.} R. Kralovics, F. Passamonti, A. S. Buser, S. S. Teo, R. Tiedt, J. R. Passweg, A. Tichelli, M. Cazzola and R. C. Skoda, “A gain-of-function mutation of JAK2 in myeloproliferative disorders,” N. Engl J. Med., 352:1779-1790, 2005.
^{4.} R. L. Levine, M. Loriaux, B. J. Huntly, M. Loh, M. Beran, E. Stoffregen, R. Berger, J. J. Clark, S. G. Willis, K. Nguyen, N. Flores, E. Estey, N. Gattermann, S. Armstrong, T. A. Look, J. D. Griffin, O. A. Bernard, D. G. Gilliland, B. J. Druker and M. W. Deininger, “The JAK2V617F activating mutation occurs in chronic myelomonocytic leukemia and acute myeloid leukemia, but not in acute lymphoblastic leukemia or chronic lymphocytic leukemia,” Blood, 106(10):3377-9, 2005.
^{5.} D. P. Steensma, G. W. Dewald, T. L. Lasho, H. L. Powell, R. F. McClure, R. L. Levine, D. G. Gilliland and A. Tefferi, “The JAK2 V617F activating tyrosine kinase mutation is an infrequent event in both “atypical” myeloproliferative disorders and myelodysplastic syndromes,” Blood, 106:1207-1209, 2005.
^{6.} Z. J. Zhao, W. Vainchenker, S. B. Krantz, N. Casadevall and S. N. Constantinescu, “Role of tyrosine kinases and phosphatases in polycythemia vera,” Semin. Hematol., 42:221-229, 2005.
^{7.} A. V. Jones, S. Kreil, K. Zoi, K. Waghom, C. Curtis, L. Zhang, J. Score, R. Seear, A. J. Chase, F. H. Grand, H. White, C. Zoi, D. Loukopoulos, E. Terpos, E. C. Vervessou, B. Schultheis, M. Emig, T. Ernst, E. Lengfelder, R. Hehlmann, A. Hochhaus, D. Oscier, R. T. Silver, A. Reiter and N. C. Cross, “Widespread occurrence of the JAK2 V617F mutation in chronic myeloproliferative disorders,” Blood, 106:2162-2168, 2005.
^{8.} A. J. Bench and H. L. Pahl, “Chromosomal Abnormalities and Molecular Markers in Myeloproliferative Disorders,” Semin. Hematol., 42:196-205, 2005.
^{9.} L. M. Scott, P. J. Campbell, E. J. Baxter, T. Todd, P. Stephens, S. Edkins, R. Wooster, M. R. Stratton, P. A. Futreal and A. R. Green, “The V617F JAK2 mutation is uncommon in cancers and in myeloid malignancies other than the classic myeloproliferative disorders,” Blood, 106:2920-2921, 2005.
^{10.} S. Sulong, M. Case, L. Minto, B. Wilkins, A. Hall and J. Irving, “The V617F mutation in Jak2 is not found in childhood acute lymphoblastic leukaemia,” Br. J. Haematol., 130:964-965, 2005.
^{11.} A. Tefferi, T. L. Lasho, S. M. Schwager, D. P. Steensma, R. A. Mesa, C. Y. Li, M. Wadleigh and D. Gary Gilliland, “The JAK2 tyrosine kinase mutation in myelofibrosis with myeloid metaplasia: lineage specificity and clinical correlates,” Br. J. Haematol., 131:320-328, 2005.
^{12.} C. C. Chang, J. Lorek, D. E. Sabath, Y. Li, C. R. Chitambar, B. Logan, B. Kampalath and R. P. Cleveland, “Expression of MUM1/IRF4 correlates with clinical outcome in patients with B-cell chronic lymphocytic leukemia,” Blood, 100:4671-4675, 2002.
^{13.} D. Xu, J. Du, C. Schultz, A. Ali and H. Ratech, “Rapid and accurate detection of monoclonal immunoglobulin heavy chain gene rearrangement by DNA melting curve analysis in the LightCycler System,” J. Mol. Diagn., 4:216-222, 2002.
^{14.} J. Thiele and H. M. Kvasnicka, “Chronic myeloproliferative disorders with thrombocythemia: a comparative study of two classification systems (PVSG, WHO) on 839 patients,” Ann. Hematol., 82:148-152, 2003.
^{15.} J. J. Michiels, “Clinical, pathological and molecular features of the chronic myeloproliferative disorders: MPD 2005 and beyond,” Hematology, 10(Suppl 1):215-223, 2005.
^{16.} J. J. Michiels and J. Thiele, “Clinical and pathological criteria for the diagnosis of essential thrombocythemia, polycythemia vera, and idiopathic myelofibrosis (agnogenic myeloid metaplasia),” Int. J. Hematol., 76:133-145, 2002.
^{17.} T. L. Lasho, R. Mesa, D. G. Gilliland and A. Tefferi, “Mutation studies in CD3+, CD19+ and CD34+ cell fractions in myeloproliferative disorders with homozygous JAK2(V617F) in granulocytes,” Br. J. Haemnatol., 130:797-799, 2005.
^{18.} K. Shannon and R. A. Van Etten, “JAKing up hematopoietic proliferation,” Cancer Cell 7:291-293, 2005.
^{19.} S. B. Needleman and C. D. Wunsch, “A general method applicable to the search for similarities in the amino acid sequence of two proteins”; J. Mol. Biol. 1970 March; 48(3):443-53.
^{20.} M. Gribskov, R. R. Burgess, and J. Devereux, “PEPPLOT, a protein secondary structure analysis program for the UWGCG sequence analysis software package”; Nucleic Acids Res.” 1986 Jan 10; 14(1):327-34.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for detecting the presence or absence of a JAK2 mutation in a population of polynucleotides, said method comprising:
(a) performing at least one cycling step, wherein said cycling step comprises at least a first amplifying step and at least a first hybridizing step, wherein said at least a first amplifying step comprises contacting said sample with a pair of JAK2-specific primers to produce a JAK2 amplification product if a JAK2 nucleic acid molecule is present in said sample, wherein said at least a first hybridizing step comprises contacting said sample with a pair of JAK2-specific probes, wherein the members of said pair of JAK2-specific probes hybridize within no more than about five nucleotides of each other, wherein the first member of said pair of JAK2-specific probes is labeled with a donor fluorescent moiety and the second member of said pair of JAK2-specific probes is labeled with a corresponding acceptor fluorescent moiety; and
(b) detecting the presence or absence of fluorescence resonance energy transfer (FRET) between said donor fluorescent moiety of said first member of said pair of JAK2-specific probes and said acceptor fluorescent moiety of said second member of said pair of JAK2-specific probes, wherein the presence of FRET is indicative of the presence of one or more JAK2-containing polynucleotides in said population, and wherein the absence of FRET is indicative of the absence of a JAK2-containing polynucleotide in said population.

2. The method of claim 1, wherein said pair of JAK2-specific primers comprises a first oligonucleotide primer of less than about 50 nucleotides in length, and further wherein said first oligonucleotide primer comprises a nucleic acid sequence selected from the group consisting of SEQ ID NO:1, SEQ ID NO:2, SEQ ID NO:3, and SEQ ID NO:4.

3. The method of claim 2, wherein said pair of JAK2-specific primers comprises a first oligonucleotide primer of less than about 50 nucleotides in length, and further wherein said first oligonucleotide primer comprises the nucleic acid sequence of SEQ ID NO:1.

4. The method of claim 1, wherein said pair of JAK2-specific primers comprises a second oligonucleotide primer of less than about 50 nucleotides in length, and further wherein said second oligonucleotide primer comprises a nucleic acid sequence selected from the group consisting of SEQ ID NO:5, SEQ ID NO:6, SEQ ID NO:7, SEQ ID NO:8, and SEQ ID NO:9.

5. The method of claim 4, wherein said pair of JAK2-specific primers comprises a second oligonucleotide primer of less than about 50 nucleotides in length, and further wherein said second oligonucleotide primer comprises the nucleic acid sequence of SEQ ID NO:5.

6. The method of claim 1, wherein said pair of JAK2-specific primers comprises a first oligonucleotide primer of less than about 50 nucleotides in length that comprises the nucleic acid sequence of SEQ ID NO:1, and a second oligonucleotide primer of less than about 50 nucleotides in length that comprises the nucleic acid sequence of SEQ ID NO:5.

7. The method of claim 1, wherein said pair of JAK2-specific detection probes comprises a first oligonucleotide probe of less than about 50 nucleotides in length, and further wherein said first oligonucleotide probe comprises a nucleic acid sequence selected from the group consisting of SEQ ID NO:10, SEQ ID NO:11, SEQ ID NO:12, SEQ ID NO:13, and SEQ ID NO:14.

8. The method of claim 7, wherein said pair of JAK2-specific detection probes comprises a first oligonucleotide probe of less than about 50 nucleotides in length, and further wherein said first oligonucleotide probe comprises the nucleic acid sequence of SEQ ID NO:10.

9. The method of claim 8, wherein said pair of JAK2-specific detection probes comprises a second oligonucleotide probe of less than about 50 nucleotides in length, and further wherein said second oligonucleotide probe comprises a nucleic acid sequence selected from the group consisting of SEQ ID NO:15, SEQ ID NO:16, SEQ ID NO:17, SEQ ID NO:18, SEQ ID NO:19, SEQ ID NO:20, SEQ ID NO:21,
SEQ ID NO:22, and SEQ ID NO:23.

10. The method of claim 9, wherein said pair of JAK2-specific detection probes comprises a second oligonucleotide probe of less than about 50 nucleotides in length, and further wherein said second oligonucleotide probe comprises the nucleic acid sequence of SEQ ID NO:15.

11. The method of claim 1, wherein said pair of JAK2-specific primers comprises a first oligonucleotide primer of less than about 50 nucleotides in length that comprises the nucleic acid sequence of SEQ ID NO:1, and a second oligonucleotide primer of less than about 50 nucleotides in length that comprises the nucleic acid sequence of SEQ ID NO:5.

12. The method of claim 1, wherein said pair of JAK2-specific detection probes comprises a first oligonucleotide probe of less than about 50 nucleotides in length that comprises the nucleic acid sequence of SEQ ID NO:10, and a second oligonucleotide probe of less than about 50 nucleotides in length that comprises the nucleic acid sequence of SEQ ID NO:15.

13. The method of claim 1, wherein said pair of JAK2-specific primers comprises a first oligonucleotide primer of less than about 50 nucleotides in length that comprises the nucleic acid sequence of SEQ ID NO:1, and a second oligonucleotide primer of less than about 50 nucleotides in length that comprises the sequence of SEQ ID NO:5; and further wherein said JAK2-specific detection probes comprises a first oligonucleotide probe of less than about 50 nucleotides in length that comprises the nucleic acid sequence of SEQ ID NO:10, and a second oligonucleotide probe of less than about 50 nucleotides in length that comprises the nucleic acid sequence of SEQ ID NO:15.

14. The method of claim 1, wherein said members of said pair of JAK2-specific probes, hybridize within no more than about one or two nucleotides of each other.

15. The method of claim 1, wherein said population of polynucleotides is obtained from a biological sample from an individual.

16. The method of claim 1, wherein said first cycling step comprises contacting said population of polynucleotides with a pair of JAK2^{V617F}-specific primers to produce a JAK2^{V617F} amplification product if a JAK2^{V617F}-encoding nucleic acid molecule is present in said population of polynucleotides, and further wherein said at least a first hybridizing step comprises contacting said population of polynucleotides with a pair of JAK2 6^{V617F}-specific probes, wherein the members of said pair of JAK2^{V617F}-specific probes hybridize within no more than about five nucleotides of each other, wherein the first member of said pair of JAK2^{V617F}-specific probes is labeled with a donor fluorescent moiety and the second member of said pair of JAK2^{V617F}-specific probes is labeled with a corresponding acceptor fluorescent moiety.

17. The method of claim 16, wherein said donor fluorescent moiety is fluorescein.

18. The method of claim 16, wherein said corresponding acceptor fluorescent moiety is selected from the group consisting of LC-Red 640, LC-Red 705, Cy5, and Cy5.5.

19. The method of claim 16, wherein said detecting step comprises exciting said sample at a wavelength absorbed by said donor fluorescent moiety and visualizing and/or measuring the wavelength emitted by said acceptor fluorescent moiety.

20. The method of claim 16, wherein said detecting comprises quantitating said FRET.

21. The method of claim 16, wherein said detecting step is performed after each cycling step.

22. The method of claim 16, wherein said detecting step is performed in real time.

23. The method of claim 16, further comprising the additional step of determining the melting temperature between one or both of said JAK2-specific probe(s) and said JAK2 amplification product, wherein said melting temperature confirms said presence or said absence of said JAK2-containing polynucleotide in said population of polynucleotides.

24. The method of claim 16, wherein the presence of said FRET within about 60 cycling steps is indicative of the presence of a JAK2-containing polynucleotide in said population of polynucleotides.

25. The method of claim 24, wherein the presence of said FRET within about 50 cycling steps is indicative of the presence of a JAK2-containing polynucleotide in said population of polynucleotides.

26. The method of claim 25, wherein the presence of said FRET within about 40 cycling steps is indicative of the presence of a JAK2-containing polynucleotide in said population of polynucleotides.

27. The method of claim 26, wherein the presence of said FRET within about 30 cycling steps is indicative of the presence of a JAK2-containing polynucleotide in said population of polynucleotides.

28. The method of claim 16, wherein said biological sample is selected from the group consisting of blood, plasma, cells, tissues, and serum.

29. The method of claim 16, wherein said cycling step is further performed on a control sample.

30. The method of claim 29, wherein said control sample comprises at least a portion of a known JAK2 polynucleotide sequence.

31. A mammalian JAK2^{V617F}-specific oligonucleotide amplification primer set, wherein the first amplification primer is less than about 50 nucleotides in length and comprises the nucleotide sequence of SEQ ID NO:1 and the second amplification primer is less than about 50 nucleotides in length and comprises the nucleotide sequence of SEQ ID NO:5.

32. The mammalian JAK2^{V617F}-specific oligonucleotide amplification primer set of claim 31, wherein the first amplification primer is less than about 40 nucleotides in length and comprises the nucleotide sequence of SEQ ID NO:1 and the second amplification primer is less than about 40 nucleotides in length and comprises the nucleotide sequence of SEQ ID NO:5.

33. The mammalian JAK2^{V617F}-specific oligonucleotide amplification primer set of claim 32, wherein the first amplification primer is less than about 30 nucleotides in length and comprises the nucleotide sequence of SEQ ID NO:1 and the second amplification primer is less than about 30 nucleotides in length and comprises the nucleotide sequence of SEQ ID NO:5.

34. The mammalian JAK2 ^{V617F}-specific oligonucleotide amplification primer set of claim 33, wherein the first amplification primer consists essentially of the nucleotide sequence of SEQ ID NO:1 and the second amplification primer consists essentially of the nucleotide sequence of SEQ ID NO:5.

35. A mammalian JAK2^{V617F}-specific oligonucleotide detection probe set, wherein the first detection probe is less than about 50 nucleotides in length and comprises the nucleotide sequence of SEQ ID NO:10 and the second detection probe is less than about 50 nucleotides in length and comprises the nucleotide sequence of SEQ ID NO:15.

36. The mammalian JAK2^{V617F}-specific oligonucleotide detection probe set of claim 35, wherein the first detection probe is less than about 40 nucleotides in length and comprises the nucleotide sequence of SEQ ID NO:10 and the second detection probe is less than about 40 nucleotides in length and comprises the nucleotide sequence of SEQ ID NO:15.

37. The mammalian JAK2^{V617F}-specific oligonucleotide detection probe set of claim 36, wherein the first detection probe is less than about 30 nucleotides in length and comprises the nucleotide sequence of SEQ ID NO:10 and the second detection probe is less than about 30 nucleotides in length and comprises the nucleotide sequence of SEQ ID NO:15.

38. The mammalian JAK2^{V617F}-specific oligonucleotide detection probe set of claim 37, wherein the first detection probe consists essentially of the nucleotide sequence of SEQ ID NO:10 and the second detection probe consists essentially of the nucleotide sequence of SEQ ID NO:15.

39. A kit comprising, in suitable container means, the oligonucleotide amplification primer set of claim 31, and instructions for using said primer set in a PCR amplification of a JAK2-containing polynucleotide.

40. The kit of claim 39, further comprising, in suitable container means, the oligonucleotide detection probe set of claim 35, and instructions for using said probe set in a FRET detection assay.

41. The kit of claim 40, further comprising instructions for using said probe and said primer sets in the detection of a polynucleotide encoding a JAK2^{V617F} mutation using a real-time PCR-FRET microvolume fluorometric analysis.

42. An article of manufacture, comprising: a pair of JAK2-specific amplification primers; a pair of JAK2-specific detection probes; at least one donor fluorescent moiety, and at least one corresponding acceptor fluorescent moiety.

43. The article of manufacture of claim 42, wherein said at least one donor fluorescent moiety is operably linked to a first member of said pair of JAK2-specific detection primers; and wherein said at least one corresponding acceptor fluorescent moiety is operably linked to a second member of said pair of JAK2-specific detection primers.

44. The article of manufacture of claim 43, comprising: a pair of JAK2^{V617F}-specific amplification primers; a pair of JAK2^{V617F}-specific detection probes; at least one donor fluorescent moiety, and at least one corresponding acceptor fluorescent moiety.

45. The article of manufacture of claim 44, wherein said at least one donor fluorescent moiety is operably linked to a first member of said pair of JAK2^{V617F}-specific detection primers; and wherein said at least one corresponding acceptor fluorescent moiety is operably linked to a second member of said pair of JAK2^{V617F}-specific detection primers.

46. The article of manufacture of claim 45, further comprising a package insert having instructions for using said pair of primers and said pair of probes to detect the presence or absence of a polynucleotide encoding a JAK2^{V617F} mutant polypeptide in a population of polynucleotides obtained from a biological sample of a human.

47. A composition comprising:
(a) a pair of JAK2-specific amplification primers; and
(b) a pair of JAK2-specific FRET detection probes; wherein a first member of said pair of JAK2-specific FRET detection probes is operably linked to at least one donor fluorescent moiety, and wherein a second member of said pair of JAK2-specific FRET detection probes is operably linked to at least one corresponding acceptor fluorescent moiety.

48. The composition of claim 47, wherein said donor fluorescent moiety is fluoroscein, and wherein said corresponding acceptor fluorescent moiety is selected from the group consisting of LightCycler Red 610, LightCycler 640, LightCycler 670, and LightCycler 705.

49. The composition of claim 48, wherein at least one member of said pair of JAK2-specific FRET detection probes is blocked at its 3′-hydroxyl end with a phosphate group to prevent polymerase extension from said member.

50. A composition comprising a pair of JAK2-specific amplification primers, wherein said pair of JAK2-specific primers comprises:
(a) a first oligonucleotide primer of less than about 50 nucleotides in length, wherein said first oligonucleotide primer comprises a nucleic acid sequence selected from the group consisting of SEQ ID NO:1, SEQ ID NO:2, SEQ ID NO:3, and SEQ ID NO:4; and
(b) a second oligonucleotide primer of less than about 50 nucleotides in length, wherein said second oligonucleotide primer comprises a nucleic acid sequence selected from the group consisting of SEQ ID NO:5, SEQ ID NO:6, SEQ ID NO:7, SEQ ID NO:8, and SEQ ID NO:9.

51. The composition of claim 50, further comprising a pair of JAK2^{V617F}-specific oligonucleotide detection probes, wherein said pair of JAK2^{V617F}-specific oligonucleotide detection probes comprises:
(a) a first oligonucleotide probe of less than about 50 nucleotides in length, wherein said first oligonucleotide probe comprises a nucleic acid sequence selected from the group consisting of SEQ ID NO:10, SEQ ID NO:11, SEQ ID NO:12, SEQ ID NO:13, and SEQ ID NO:14; and
(b) a second oligonucleotide probe of less than about 50 nucleotides in length, wherein said second oligonucleotide probe comprises a nucleic acid sequence selected from the group consisting of SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

52. A system for amplification and detection of a polynucleotide encoding a mammalian JAK2^{V617F} polypeptide, said system comprising:
(a) a pair of amplification primers, said pair comprising:
(i) a first oligonucleotide primer of less than about 50 nucleotides in length, wherein said first oligonucleotide primer comprises a nucleic acid sequence selected from the group consisting of SEQ ID NO:1, SEQ ID NO:2, SEQ ID NO:3, and SEQ ID NO:4; and
(ii) a second oligonucleotide primer of less than about 50 nucleotides in length, wherein said second oligonucleotide primer comprises a nucleic acid sequence selected from the group consisting of SEQ ID NO:5, SEQ ID NO:6, SEQ ID NO:7, SEQ ID NO:8, and SEQ ID NO:9;
(b) a pair of JAK2^{V617F}-specific oligonucleotide detection probes, said pair comprising:
(i) a first oligonucleotide probe of less than about 50 nucleotides in length, wherein said first oligonucleotide probe comprises a nucleic acid sequence selected from the group consisting of SEQ ID NO:10, SEQ ID NO:11, SEQ ID NO:12, SEQ ID NO:13, and SEQ ID NO:14;. and
(ii) a second oligonucleotide probe of less than about 50 nucleotides in length, wherein said second oligonucleotide probe comprises a nucleic acid sequence selected from the group consisting of SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

53. The system of claim 52, wherein said polynucleotide is amplified from a popoluation of nucleic acids obtained from a biological sample.

54. The system of claim 53, wherein said polynucleotide is amplified from a popoluation of nucleic acids obtained from a human blood or tissue sample.
